(19) United States
(12) Patent Application Publication
(10) Pub. No.: US 2026/0013804 A1
(43) Pub. Date: Jan. 15, 2026

(54) ADAPTIVE ALARMS BASED ON PATIENT CHARACTERISTICS

(71) Applicant: Stryker Corporation, Portage, MI (US)

(72) Inventors: Sophie Francis, Carnation, WA (US); Dara Friedman, Redmond, WA (US); McKenzie Oster, Kirkland, WA (US); Kincade Moran, Kirkland, WA (US); Nolan Frank Dost, Woodinville, WA (US); Franco Gopalakrishnan, Bothell, WA (US); Megan Cote, Redmond, WA (US); Tyler Marshall, Bothell, WA (US); Taylor Christian Sears, Bellingham, WA (US); Tom Hiblar, Lake Stevens, WA (US); Trenton Roeber, Carnation, WA (US); Kai Nellermoe, Kirkland, WA (US)

(73) Assignee: Stryker Corporation, Portage, MI (US)

(21) Appl. No.: 19/266,427

(22) Filed: Jul. 11, 2025

Related U.S. Application Data
(60) Provisional application No. 63/670,026, filed on Jul. 11, 2024.

(51) Int. Cl. A61B 5/00; A61B 7/00; G16H 10/60; G16H 40/67

(52) U.S. Cl. CPC A61B 5/746; A61B 5/7405; A61B 5/742; A61B 7/003; G16H 10/60; G16H 40/67; A61B 2560/0247; A61B 2560/0252

(57) ABSTRACT

Techniques for mitigating alarm fatigue are described. In the examples described herein, the alarms of a medical device are customized to a patient who is located at an emergency scene. An example method includes receiving, by a medical device, contextual data associated with an emergency scene where a patient is located, determining, by the medical device analyzing the contextual data, a characteristic of the patient, customizing, by the medical device, a set of alarms to the characteristic of the patient to obtain a customized set of alarms, generating, by the medical device analyzing a physiological parameter of the patient, an alarm of the customized set of alarms, and causing, by the medical device, the alarm to be output.

### CROSS-REFERENCE TO RELATED APPLICATION

This application claims the benefit of U.S. Provisional Application No. 63/670,026, titled “ADAPTIVE ALARMS BASED ON PATIENT CHARACTERISTICS”, and filed on Jul. 11, 2024, which is incorporated by reference herein in its entirety.

### BACKGROUND

Medical devices can be used to facilitate patient monitoring, facilitate patient treatments, or a combination of both. Many medical devices are also configured to output alarms that are intended to alert a caretaker with respect to a medical condition of the patient. At an emergency scene, several medical devices may be used contemporaneously to monitor and/or treat a patient, and each of these medical devices can potentially output several different alarms. In these scenarios, caretakers can be exposed to an excessive number of alarms, which can result in desensitization to the alarms and an increased rate of missed or ignored alarms, which renders the alarms ineffective. This type of sensory overload is also referred to as “alarm fatigue.”

### BRIEF DESCRIPTION OF THE DRAWINGS

FIG. 1A illustrates a medical device at an emergency scene where a patient is located, the medical device configured to customize its alarms to a characteristic of the patient, the characteristic having been determined from context of the emergency scene.

FIG. 1B illustrates a medical device at an emergency scene where a patient is located, the medical device configured to customize its alarms to a physiological parameter of the patient, the physiological parameter having been determined from device data the medical device received from a patient device of the patient.

FIG. 2 illustrates an example medical device configured to determine, among other things, that a patient is likely experiencing a respiratory problem by processing audio data representing a sound(s) in an environment of the medical device, and to customize a set of alarms of the medical device to obtain a customized set of alarms tailored to the determined medical condition of the patient.

FIG. 3 illustrates an example process for determining a characteristic of a patient from context of an emergency scene where the patient is located, and customizing a set of alarms of a medical device to the characteristic of the patient in order to output an alarm(s) that is customized to the patient.

FIG. 4 illustrates example techniques for determining a characteristic of a patient from context of an emergency scene where the patient is located.

FIG. 5 illustrates example techniques for customizing a set of alarms.

FIG. 6 illustrates an example process for managing alarms of a medical device in the context of one or more additional medical devices in a vicinity of the medical device.

FIG. 7 illustrates an example process for managing alarms of a medical device based on a medical history of a patient at an emergency scene.

FIG. 8 illustrates an example process for preconfiguring the alarms of a medical device and simulating the alarms via an alarm configuration user interface presented on a user device.

FIG. 9 illustrates an example process for determining a physiological parameter of a patient from device data received by a medical device from a patient device of the patient, and customizing a set of alarms of the medical device to the physiological parameter of the patient in order to output an alarm(s) that is customized to the patient.

FIG. 10 illustrates an example of an external defibrillator configured to perform various functions described herein.

### DETAILED DESCRIPTION

Various implementations described herein relate to techniques for mitigating alarm fatigue by adapting the alarms of a medical device so that the alarms are customized to a patient who is located at an emergency scene. In some examples, one or more rescuers arrive at the emergency scene with a medical device that is to be used for monitoring and/or treating the patient. In some scenarios, the rescuer(s), upon arriving at the emergency scene, has no prior knowledge of who the patient is, let alone the patient's medical history. Moreover, at the time of arrival, the rescuer(s) may not know the current medical condition of the patient and/or what type of treatment(s) might help the patient. In some examples, the emergency scene is at a public location with many bystanders, ambient noise, and/or distractions that can potentially detract a rescuer's attention from the patient who is in need of medical care. In some examples, multiple medical devices are managing care of the same patient, each with its own set of alarms that are intended to alert the rescuer(s) of potential danger with respect to a medical condition of the patient. These types of settings can cause sensory overload for the rescuer(s) at the emergency scene. The techniques, devices, and systems described herein address this problem by adapting the alarms of a medical device so that the alarms are customized to the patient, thereby mitigating alarm fatigue and rendering the alarms more effective in alerting a rescuer(s).

According to various implementations of the present disclosure, a medical device is configured to customize a set of alarms to a characteristic of a patient, wherein the characteristic is determined from context of an emergency scene where the patient is located. For example, the medical device may include an input device(s) configured to receive contextual data associated with the emergency scene. This contextual data can take many forms, such as location data, audio data, image data, environmental data, and/or the like. To illustrate, consider an example where the contextual data includes audio data. In this example, the medical device may be equipped with a microphone(s), which is an example of an input device. The microphone(s) may capture a sound(s) in an environment of the medical device while the medical device is located at the emergency scene, and audio data representing that sound(s) may be generated. A processor(s) of the medical device can analyze this audio data to determine (e.g., infer) a characteristic of the patient. Consider an example where the audio data is processed to determine a sound recognition result indicative of agonal breathing. In this example, the characteristic determined from context of the emergency scene may be that the patient is likely experiencing a respiratory-related medical condition. Although the contextual data includes audio data in this example, it is to be appreciated that other types of contextual data (e.g., location data, image data, environmental data, etc.) may be analyzed to infer the same patient characteristic and/or other types of patient characteristics, as described in the various examples herein. Once the characteristic of the patient is determined from the context of the emergency scene, the processor(s) of the medical device may customize a set of alarms to the determined patient characteristic to obtain a customized set of alarms that is tailored to the patient.

Various ways of customizing alarms are described herein. In one example, the set of alarms may be customized by prioritizing the output of certain alarms over others. In the example described above, respiratory-related alarms may be prioritized over other alarms that are unrelated to a respiratory medical condition. In another example, the limits (e.g., high limits and/or low limits) associated with certain alarms may be customized by changing the threshold(s) used for generating those alarms. The customized set of alarms is better tailored to the characteristic of the patient, which renders the alarms of the medical device more effective in alerting a rescuer(s) at the emergency scene, which leads to improved patient outcomes. For example, alarms that are deprioritized may be disabled or muted in order to direct the rescuer's attention to the prioritized alarms when those prioritized alarms are generated, and/or the customized alarms may be more or less sensitive to certain medical conditions in order to alert the rescuer(s) only when the rescuer(s) needs to be alerted, and/or by refraining from alerting the rescuer(s) at times when it is unnecessary to do so.

In some examples, the medical device described herein is configured to receive, from a patient device of a patient located at an emergency scene, device data indicative of a physiological parameter of the patient, and to customize a set of alarms to the physiological parameter of the patient. To illustrate, consider an example where the patient device is a wearable device, such as a smart watch worn on the patient's wrist and configured to monitor the heart rate of the patient. In this example, the medical device may receive, from the patient device, device data indicating a heart rate of the patient. By analyzing this device data, the medical device may determine that the patient's heart rate is elevated and that the patient is likely experiencing a cardiac event (e.g., ventricular fibrillation (VF)). It is to be appreciated, however, that the device data may indicate other types of physiological parameters (e.g., body temperature, blood pressure, blood oxygenation, etc.), which may be indicative of other types of medical conditions, as described in the various examples herein. Once the physiological parameter of the patient is determined from the received device data, the processor(s) of the medical device may customize a set of alarms to the determined physiological parameter to obtain a customized set of alarms that is tailored to the patient. The alarms may be customized in similar ways to those described above and elsewhere in this disclosure.

It is to be appreciated that, while several of the examples described herein pertain to a medical device implemented as a monitor-defibrillator (e.g., external defibrillator), the techniques described herein may be implemented with respect to other types of medical devices. Moreover, while several of the examples described herein pertain to an emergency scene that is outside of a hospital setting, the techniques described herein may be equally useful for mitigating alarm fatigue in-hospital settings, such as when a patient initially arrives at a hospital and is taken to an emergency room, which is a notoriously chaotic environment.

Implementations of the present disclosure are directed to improvements in the technical field of medical devices. With conventional medical devices, caretakers, such as rescuers who arrive at an emergency scene where a patient is located, may experience alarm fatigue due to their exposure to an excessive number of alarms output by the medical devices. The techniques, devices, and systems described herein mitigate alarm fatigue by adapting the alarms of a medical device so that the alarms are customized to a patient who is located at an emergency scene, thereby rendering the alarms more effective in alerting a caretaker with respect to a medical condition of the patient, which leads to improved patient outcomes.

Various examples will now be described with reference to the accompanying drawings.

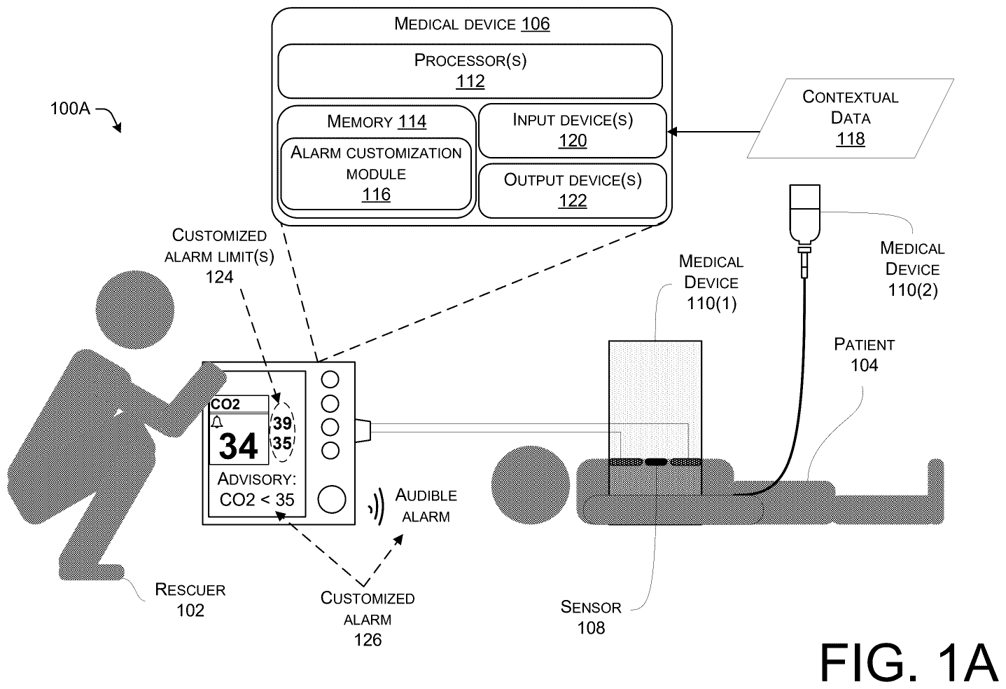

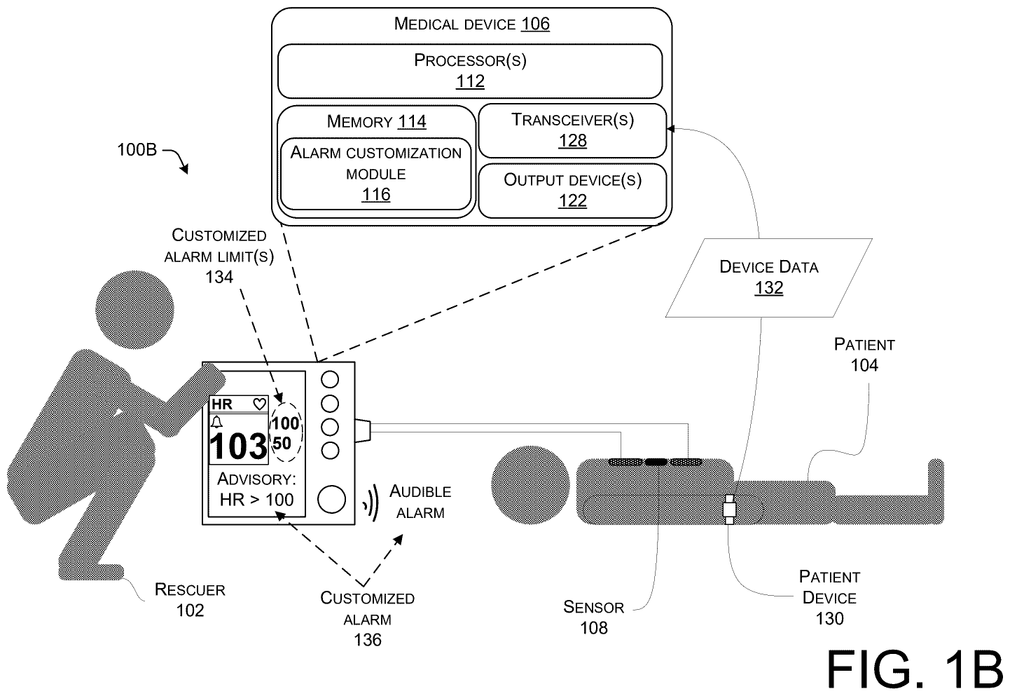

FIG. 1A illustrates an emergency scene 100A. The emergency scene 100A, in some implementations, is outside of a clinical environment, such as a public area that is located remotely from a hospital or other managed care facility. In some cases, the emergency scene 100A is within a clinical environment, such as within a hospital (e.g., in an emergency room). In various implementations, a rescuer 102 is monitoring and/or treating a patient 104 located at the emergency scene 100A. For instance, the rescuer 102 is an emergency medical technician (EMT) who has arrived at the emergency scene 100A to monitor and/or treat a medical condition of the patient 104 using one or more medical devices. In some cases, the patient 104 is experiencing cardiac arrest, a blocked airway, or some other dangerous medical condition.

As shown in FIG. 1A, the rescuer 102 is monitoring and/or treading a medical condition of the patient 104 using a medical device 106. Although the medical device 106 depicted in FIG. 1A exemplifies a monitor-defibrillator, the medical device 106 can be a medical imaging device, an ultrasound monitor, a standalone electrocardiogram (ECG) monitor, a mechanical chest compression device, an automated external defibrillator (AED), a medication-administering device, a smart bag-valve mask, a ventilator, a heart-lung machine, or another type of medical device. The medical device 106 includes and/or is communicatively coupled to a sensor 108. The sensor 108 is configured to detect at least one physiological parameter of the patient 104. Examples of physiological parameters include, for instance, an ECG, an impedance, a force administered to the patient 104, a blood pressure, an airway parameter (e.g., a partial pressure of carbon dioxide, a partial pressure of oxygen, a capnograph, an end tidal gas parameter, a flow rate, etc.), a blood oxygenation (e.g., a pulse oximetry value, a regional oximetry value, etc.), an electroencephalogram (EEG), a temperature, a heart sound, a blood flow rate, a physiological geometry (e.g., a shape of a blood vessel, an inner ear shape, etc.), a heart rate, a pulse rate, or the like. For example, the sensor 108 includes at least one of electrodes, a detection circuit, defibrillator pads, a force sensor, a blood pressure cuff, an ultrasound-based blood pressure sensor, an invasive (e.g., intra-arterial) blood pressure sensor (e.g., including a cannula inserted into the patient 104), a gas sensor (e.g., a carbon dioxide and/or oxygen sensor), a flowmeter, a pulse oximetry sensor, a regional oximetry sensor, a thermometer, a microphone, an ultrasound transducer, a medical imaging device (e.g., an ultrasound imaging device), or the like. In various cases, the medical device 106 outputs the physiological parameter(s) to the rescuer 102. For instance, the medical device 106 includes a display(s), a speaker(s), or haptic feedback device(s) that conveys the physiological parameter(s) to the rescuer 102. In the example of FIG. 1A, the medical device 106 is outputting, via a display of the medical device 106, a carbon dioxide parameter, such as an end tidal carbon dioxide (EtCO₂) parameter.

In some examples, one or more additional medical devices 110 may be collocated at the emergency scene 100A with the medical device 106. In the example of FIG. 1A, a first additional medical device 110(1) represents a mechanical chest compression device, and a second additional medical device 110(2) represents a medication-administering device, such as an intravenous (IV) fluid pump. The additional medical devices 110 are merely examples, and it is to be appreciated that other types of medical devices 110 may be in a vicinity of the medical device 106 at the emergency scene 100A, such as a monitor-defibrillator, a medical imaging device, an ultrasound monitor, a standalone ECG monitor, an AED, a smart bag-valve mask, a ventilator, a heart-lung machine, and/or the like. Examples of treatments (e.g., therapies) that can be administered by the medical device 106 and/or the additional medical device(s) 110 include defibrillation, pacing, cardioversion, administration of chest compressions, administration of oxygen to the airway of the patient 104, movement of air in the airway of the patient, administration of fluids to the patient 104, extracorporeal membrane oxygenation (ECMO), administration of a medication to the patient 104, and/or the like. In some implementations, the additional medical device(s) 110 is/are configured to detect one or more physiological parameters of the patient 104, such as one or more of the example physiological parameters described above with respect to the sensor 108.

FIG. 1A illustrates example components of the medical device 106. For example, the medical device 106 may include one or more processors 112, such as a central processing unit(s) (CPU(s)), a graphics processing unit(s) (GPU(s)), both CPU(s) and GPU(s), or another processing unit or component known in the art. The processor(s) 112 is operably connected to memory 114. In various implementations, the memory 114 is volatile (such as random access memory (RAM)), non-volatile (such as read only memory (ROM), flash memory, etc.) or some combination of the two. The memory 114 stores instructions that, when executed by the processor(s) 112, cause the processor(s) 112 to perform various operations described herein. In various examples, the memory 114 stores methods, threads, processes, applications, objects, modules, any other sort of executable instruction, or a combination thereof. An example depicted in FIG. 1A includes an alarm customization module 116 that, when executed by the processor(s) 112, causes the processor(s) 112 to, among other things, determine a characteristic of the patient 104 by analyzing contextual data 118, and customize a set of alarms to the determined patient characteristic to obtain a customized set of alarms that is tailored to the patient 104. As used herein, the term “module,” and its equivalents, refers to data including instructions that, when executed by one or more processors, cause the processor(s) to perform one or more operations. In some cases, the memory 114 stores files, databases, or a combination thereof. For example, the medical device 106 may collect physiological parameter data (e.g., CO₂ data, heart rate data, etc.) during use of the medical device 106, and this and other data may be stored, at least temporarily, in the memory 114. In some examples, the memory 114 includes RAM, ROM, electrically erasable programmable read-only memory (EEPROM), flash memory, or any other memory technology. In some examples, the memory 114 includes CD-ROMs, digital versatile discs (DVDs), content-addressable memory (CAM), and/or other optical storage, magnetic cassettes, magnetic tape, magnetic disk storage and/or other magnetic storage devices, and/or any other medium (e.g., non-transitory computer-readable medium) which can be used to store the desired information and which can be accessed by the processor(s) 112.

The medical device 106 may further include one or more input devices 120 and one or more output devices 122. The input device(s) 120 is configured to receive the aforementioned contextual data 118 associated with the emergency scene 100A where the patient 104 is located. The contextual data 118 may be indicative of a context of the emergency scene 100A, such as a location of the medical device 106, audio at the emergency scene 100A, images and/or video of the emergency scene 100A (or at least a portion of the emergency scene 100A), environmental conditions (e.g., heatwaves, air quality alerts, flooding alerts, etc.), and/or the like. Accordingly, the input device(s) 120 that is configured to receive the contextual data 118 can take many forms, such as a microphone(s), a camera(s), a touch-sensitive display(s), a keypad(s), a cursor control device(s), a location-determining device(s) (e.g., a Global Positioning System (GPS) receiver), a transceiver(s) that is configured to receive the contextual data 118 wirelessly from a nearby sending device and/or over a network(s), or any combination thereof. The output device(s) 122 includes at least one of a display(s), a light emitting element(s) (e.g., a light emitting diode(s) (LED(s))), an electrochromic material(s), a speaker(s), a haptic actuator(s), a transceiver(s), a printer(s), or any combination thereof. In the examples described herein, the output device(s) 122 is configured to output an alarm(s).

In the example of FIG. 1A, the input device(s) 120 receives contextual data 118 associated with the emergency scene 100A, and the processor(s) 112 (e.g., via execution of the alarm customization module 116) determines a characteristic(s) of the patient 104 by analyzing the contextual data 118. In some examples, the characteristic(s) of the patient 104 is inferred from the contextual data 118 in the sense that the characteristic(s) of the patient 104 is deduced from the contextual data 118 as a likely characteristic of the patient 104. In some examples, the characteristic(s) of the patient 104 is a medical condition of the patient 104. Consider an example where the processor(s) 112 determines, by analyzing the contextual data 118, that the patient 104 is likely experiencing a respiratory problem. In this example, the processor(s) 112 (e.g., via execution of the alarm customization module 116) customizes a set of alarms associated with the medical device 106 to obtain a customized set of alarms that is customized to the characteristic of the patient 104. For instance, the processor(s) 112 may change a threshold(s) used for generating a respiratory-related alarm, such as a carbon dioxide alarm. This is shown in FIG. 1A as a customized alarm limit(s) 124. That is, the high limit associated with the carbon dioxide alarm may be changed from, say, 40 millimeters of mercury (mmHg) to 39 mmHg, and/or the low limit associated with the carbon dioxide alarm may be changed from, say, 33 mmHg to 35 mmHg. In other words, the limit(s) associated with the carbon dioxide alarm may be changed from a wider range to a narrower range in order to increase the sensitivity of the carbon dioxide alarm, or vice versa in order to decrease the sensitivity of the carbon dioxide alarm. Additionally, or alternatively, the limits may be shifted to encompass a different range of values, with or without widening or narrowing the range.

After obtaining the customized set of alarms, the processor(s) 112 may be configured to monitor a physiological parameter(s) of the patient 104 to determine whether to generate an alarm(s) of the customized set of alarms. For example, the sensor 108 may detect a physiological parameter(s) of the patient 104, such as an EtCO₂ parameter, and the processor(s) 112 may receive the physiological parameter(s) via the sensor 108 and compare the physiological parameter(s) to a threshold(s) (e.g., the customized alarm limit(s) 124) associated with the customized set of alarms. In response to comparing the physiological parameter to the threshold(s), the processor(s) 112 may generate an alarm(s) of the customized set of alarms. For example, if the EtCO₂ parameter is 34 mmHg, the processor(s) 112 may determine that this EtCO₂ parameter is less than a customized threshold (e.g., the low limit of 35 mmHg) for the carbon dioxide alarm, and the processor(s) 112 may generate the carbon dioxide alarm in this scenario. The processor(s) 112 may also cause the alarm(s) to be output via the output device(s) 122. In the example of FIG. 1A, this is shown as a customized alarm 126, which is in the form of a visual alarm (e.g., “Advisory: CO_{2<35}”) presented on a display of the medical device 106 and an audible alarm (e.g., a siren, a periodic or sustained “beep” sound, etc.) output via a speaker(s) of the medical device 106. It is to be appreciated that the alarm may be output in other ways, such as a flashing or sustained LED(s) of a certain color (e.g., a red-colored, flashing LED(s)), a vibration of a haptic actuator(s), and/or the like. By customizing the alarm(s) to the characteristic of the patient 104, the alarm(s) is rendered more effective in alerting the rescuer 102 as to a current medical condition (e.g., a respiratory problem) of the patient 104.

FIG. 1B illustrates an emergency scene 100B, which may be the same or different than the emergency scene 100A described above with reference to FIG. 1A. In various implementations, a rescuer 102 is monitoring and/or treating a patient 104 located at the emergency scene 100B. For instance, the rescuer 102 is an EMT who has arrived at the emergency scene 100B to monitor and/or treat a medical condition of the patient 104 using one or more medical devices, such as the medical device 106 introduced in FIG. 1A.

In the example of FIG. 1B, the medical device 106 is shown as having a transceiver(s) 128. The transceiver(s) 128 (e.g., a wireless radio, antenna, or the like) may be configured to communicate wirelessly with one or more other devices at the emergency scene 100B, such as a patient device 130 of the patient 104, and/or the additional medical device(s) 110 described above with reference to FIG. 1A. The transceiver(s) 128 may include any sort of wireless transceivers capable of engaging in wireless communication (e.g., radio frequency (RF) communication), such as WI-FI®, WIGIG®, WIMAX®, BLUETOOTH®, near-field communication (NFC), radio frequency identification (RFID), or infrared communication.

In some cases, the monitoring and/or treatment of the patient 104 is optimized by communication between the medical device 106 and one or more other devices, such as the patient device 130. In particular examples, the transceiver(s) 128 is configured to receive, from the patient device 130, device data 132 indicative of a physiological parameter(s) of the patient 104, such as one or more of the example physiological parameters described above with respect to the sensor 108. For example, the patient device 130 may be a wearable device, such as a smart watch, that is capable of sensing a heart rate (and/or other physiological parameters) of the patient 104, and a communication signal may be received by the transceiver(s) 128, the communication signal including the device data 132 indicating the heart rate of the patient 104. Although FIG. 1B depicts an example patient device 130 in the form of a wearable smart watch, other types of patient devices 130 are contemplated herein, such as other types of wearable devices worn on other parts of the patent's body, an implantable device, a home health care device, such as an insulin pump, and/or the like. Accordingly, the device data 132 may indicate any suitable type of physiological parameter of the patient 104, such as one or more of the example physiological parameters described above with respect to the sensor 108.

To exchange data, the medical device 106 and the patient device 130 and/or an additional medical device(s) 110 are configured to establish and/or communicate via a communication channel. As used herein, the term “communication channel,” and its equivalents, may refer to a medium over which a first endpoint (e.g., a sender) transmits information to one or more second endpoints (e.g., receivers). Examples of communication channels include wired connections, such as Ethernet or fiber optic paths, as well as wireless connections, such as Institute of Electronics and Electrical Engineers (IEEE) (e.g., WI-FI, BLUETOOTH, etc.) or 3^{rd} Generation Partnership Program (3GPP) (e.g., Long Term Evolution (LTE), New Radio (NR), etc.) connections. As used herein, the term “endpoint,” and its equivalents, may refer to an entity that is configured to transmit and/or receive data. Examples of endpoints include user equipment (UE) (e.g., mobile phones, tablet computers, etc.), computers, base stations, access points (APs), servers, compute nodes, medical devices, Internet of Things (IoT) devices, and the like.

In some implementations, a communication channel between the medical device 106 and the patient device 130 is established when the medical device 106 and the patient device 130 are paired. A similar pairing procedure may be utilized between the medical device 106 and an additional medical device(s) 110, and/or between the patient device 130 and an additional medical device(s) 110. In particular cases, two devices (e.g., the medical device 106 and the patient device 130) refrain from sharing substantive data (e.g., the device data 132, physiological metrics, reports about the patient 104, instructions for treating the patient 104, etc.) until the two devices are paired. As used herein, the term “paired,” and its equivalents, may refer to a state of multiple devices that have a shared link key that enables each device to cryptographically authenticate data it receives from any other device among the multiple devices. In some examples, the medical device 106 may send, to the patient device 130, via the transceiver(s) 128, authentication data for authenticating the medical device 106 prior to receiving the device data 132. In this manner, the patient device 130 may refrain from sending the device data 132 to the medical device 106 until the medical device 106 authenticates with the patient device 130. In some examples, the authentication data may be sent to the patient device 130 as part of a challenge/response type authentication using a shared secret, such as a lengthy binary number, hexadecimal number, or other alphanumeric value sufficiently distinct that it is computationally difficult to predict. In some examples, a hashing algorithm is used to generate an authentication code based at least in part on a seed value provided in the authentication data. It is to be appreciated that these are merely example techniques for authenticating the medical device 106, and other authentication techniques may be used herein.

Various mechanisms can be utilized to pair two devices, such as the medical device 106 and the patient device 130. For example, automated pairing may involve the exchange of data and/or pairing requests/responses between the devices 106 and 130. As another example, the medical device 106 may receive an input signal from an operator (e.g., the rescuer 102) that selects the patient device 130 as a device to pair with the medical device 106, or vice versa. In some cases, the medical device 106 detects an alternative signal (e.g., a flashing light pattern) from the patient device 130 that is indicated in a pairing request. In some cases, the medical device 106 and the patient device 130 are paired, at least in part, based on signaling to and/or from an intermediary device (not illustrated in FIG. 1B). In a particular example, two or more devices can be brought into proximity to (e.g., into contact with) each other in order to facilitating pairing the medical device 106 with the patent device 130. For example, a “tap-to-pair” functionality may allow a user to bring the patient device 130 (or a component thereof) into close proximity to (e.g., into contact with) the medical device 106 (or a component thereof), or vice versa, and a short-range wireless protocol, such as BLUETOOTH, NFC, or the like, may be used to detect that the devices 106 and 130 are within a threshold distance of each other, and, in response, the devices 106 and 130 may be paired. In some cases, an intermediary device, such as a phone, may be brought into close proximity to (e.g., into contact with) the medical device 106 and/or the patent device 130 (e.g., by touching the intermediary device to both devices 106 and 130 sequentially), and a short-range wireless protocol may be used to detect these proximity events involving the intermediary device, and, in response, the devices 106 and 130 may be paired.

In particular cases, a first paired device encrypts data prior to transmitting the data to a second paired device, and the second paired device restores the original data by decrypting the encrypted data. As used herein, the term “encrypt,” and its equivalents, refers to a process of translating data from one format (e.g., an unencoded format) into an encoded format. In various cases, the encoded format is referred to as “ciphertext.” Unencoded data, which has not been encrypted, may be referred to as being in “plaintext.” In various examples, an entity encrypts data using at least one encryption key. An encryption key is a parameter that defines the translation of data from the one format into the encoded format. As used herein, the term “decrypt,” and its equivalents, refers to a process of translating data from an encoded format into another format (e.g., an unencoded format), such as a plaintext format. In various examples, an entity encrypts data using at least one decryption key. A decryption key is a parameter that defines the translation of data from the encoded format into the other format. A link key, for example, is an encryption and/or decryption key.

Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different. Cryptographic hash functions (CHFs) are examples of cryptographic techniques. Examples of cryptographic techniques include the Data Encryption Standard (DES), Advanced Encryption Standard (AES), Elliptic Curve Cryptography (ECC), Rivest-Shamir-Adleman (RSA), Secure Hash Algorithm (SHA)-1, SHA-2, SHA-3, BLAKE, BLAKE2, BLAKE3, WHIRLPOOL, MD2, MD4, MD5, MD6, Temporal Key Integrity Protocol (TKIP), Rivest cipher 4 (RC4), variably modified permutation composition (VMPC), blowfish, Twofish, Threefish, Tiny Encryption Algorithm (TEA), Extended TEA (XTEA), Corrected Block TEA (XXTEA), Diffie-Hellman exchange (DHE), elliptic curve DHE, supersingular isogeny Diffie-Hellman (SIDH) key exchange, and so on. Any suitable encryption or decryption technique can be used in accordance with implementations of this disclosure. Accordingly, in some examples, the device data 132 is encrypted and transmitted to the medical device 106, and the processor(s) 112 is configured to decrypt the device data 132 prior to customizing a set of alarms.

In the example of FIG. 1B, the transceiver(s) 128 receives, from the patient device 130, device data 132 indicative of a physiological parameter(s) of the patient 104, and the processor(s) 112 (e.g., via execution of the alarm customization module 116) customizes a set of alarms associated with the medical device 106 to obtain a customized set of alarms that is customized to the physiological parameter of the patient 104. Consider an example where the device data 132 indicates a heart rate of the patient 104 (e.g., a time series of heart rate data recorded over a period of time prior to receipt of the device data 132), and the processor(s) 112 changes a threshold(s) used for generating a cardiovascular-related alarm, such as a heart rate alarm, based at least in part the heart rate indicated by the device data 132. This is shown in FIG. 1B as a customized alarm limit(s) 134. That is, the high limit associated with the heart rate alarm may be changed from, say, 110 beats per minute (bpm) to 100 bpm, and/or the low limit associated with the heart rate alarm may be changed from, say, 45 bpm to 50 bpm. In other words, the limit(s) associated with the heart rate alarm may be changed from a wider range to a narrower range in order to increase the sensitivity of the heart rate alarm, or vice versa in order to decrease the sensitivity of the heart rate alarm. Additionally, or alternatively, the limits may be shifted to encompass a different range of values, with or without widening or narrowing the range.

After obtaining the customized set of alarms, the processor(s) 112 may be configured to monitor a physiological parameter(s) of the patient 104 to determine whether to generate an alarm(s) of the customized set of alarms. For example, the sensor 108 may detect a physiological parameter(s) of the patient 104, such as an ECG, and the processor(s) 112 may receive the physiological parameter(s) via the sensor 108 and compare the physiological parameter(s) to a threshold(s) (e.g., the customized alarm limit(s) 134) associated with the customized set of alarms. In response to comparing the physiological parameter to the threshold(s), the processor(s) 112 may generate an alarm(s) of the customized set of alarms. For example, if the ECG indicates a heart rate of 103 bpm, the processor(s) 112 may determine that this heart rate is greater than a customized threshold (e.g., the high limit of 100 bpm) for the heart rate alarm, and the processor(s) 112 may generate the heart rate alarm in this scenario. The processor(s) 112 may also cause the alarm(s) to be output via the output device(s) 122. In the example of FIG. 1B, this is shown as a customized alarm 136, which is in the form of a visual alarm (e.g., “Advisory: HR>100”) presented on a display of the medical device 106 and an audible alarm (e.g., a siren, a periodic or sustained “beep” sound, etc.) output via a speaker(s) of the medical device 106. It is to be appreciated that the alarm may be output in other ways, such as a flashing or sustained LED(s) of a certain color, a vibration of a haptic actuator(s), and/or the like. By customizing the alarm(s) to the physiological parameter(s) of the patient 104 indicated by the device data 132, the alarm(s) is rendered more effective in alerting the rescuer 102 as to a current medical condition (e.g., a cardiovascular problem) of the patient 104.

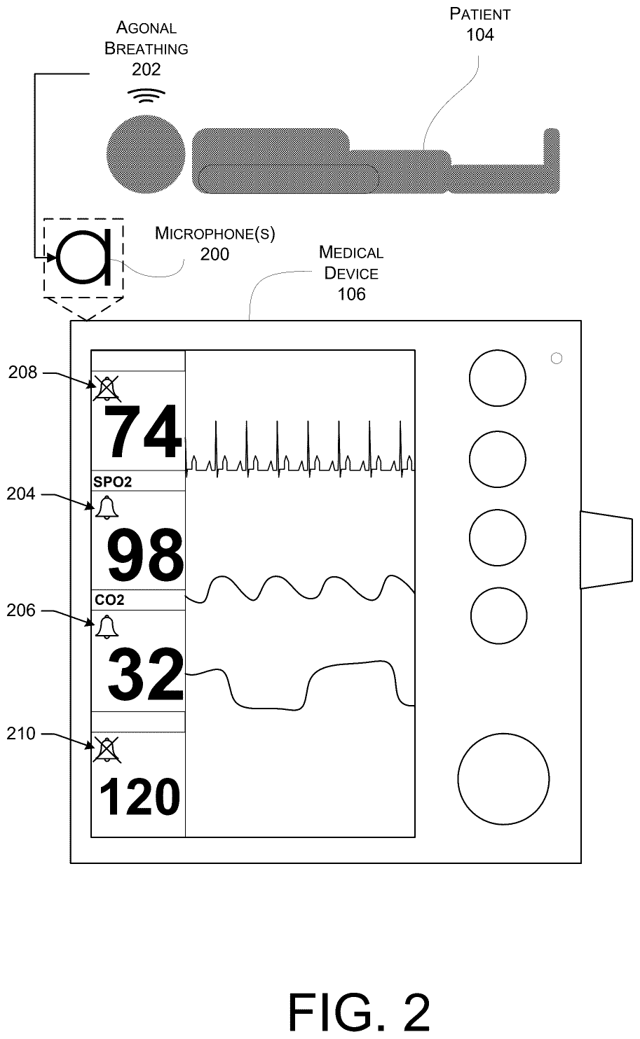

A particular example will now be described with reference to FIG. 2, which illustrates an example medical device 106 configured to determine that a patient 104 is likely experiencing a respiratory problem by processing audio data representing a sound(s) in an environment of the medical device 106, and to customize a set of alarms of the medical device 106 to obtain a customized set of alarms tailored to the determined medical condition of the patient 104. In the example of FIG. 2, the medical device 106 includes an input device 120 in the form of a microphone(s) 200, and the contextual data 118 received by this input device 120 is in the form of audio data. For example, the microphone(s) 200 may capture a sound(s) in an environment of the medical device 106 while the medical device 106 is located at an emergency scene (e.g., the emergency scene 100A), and the processor(s) 112 (e.g., via execution of the alarm customization module 116) processes the audio data to determine a sound recognition result, and determines a characteristic(s) of the patient 104 by analyzing the sound recognition result. For example, the sound recognition result may be indicative of agonal breathing 202, and the processor(s) 112 may determine, from this sound recognition result, that the patient is likely experiencing a respiratory problem. In this example, the processor(s) 112 (e.g., via execution of the alarm customization module 116) customizes a set of alarms associated with the medical device 106 to obtain a customized set of alarms that is customized to the characteristic of the patient 104. For instance, the processor(s) 112 may prioritize output of a respiratory-related alarm(s) (e.g., an oxygen saturation (SpO₂) alarm 204, a carbon dioxide alarm 206, etc.) over one or more additional alarms of the set of alarms. Additionally, or alternatively, the processor(s) 112 may disable or mute one or more additional alarms of the set of alarms that are (i) not associated with the medical condition (e.g., respiratory problem), or (ii) associated with the medical condition (e.g., respiratory problem), but not beneficial for continuing treatment of that medical condition. For example, FIG. 2 shows that a first additional alarm 208 and a second additional alarm 210 have been disabled or muted. The first additional alarm 208 might be a heart rate alarm, for example, which may not be considered to be associated with a respiratory medical condition, or which may not be beneficial for continuing treatment of the respiratory medical condition. The second additional alarm 210 might be an arterial pressure alarm, for example, which may not be considered to be associated with a respiratory medical condition, or which may not be beneficial for continuing treatment of the respiratory medical condition. Thus, disabling or muting the alarms 208 and 210 may allow the rescuer 102 to focus on the alarms 204 and/or 206 (which may have been prioritized based on the detection of agonal breathing 202).

After obtaining the customized set of alarms, the processor(s) 112 may be configured to monitor a physiological parameter(s) of the patient 104 to determine whether to generate an alarm(s) of the customized set of alarms. For example, the sensor 108 may detect a physiological parameter(s) of the patient 104, such as an EtCO₂ parameter, and the processor(s) 112 may receive the physiological parameter(s) via the sensor 108 and compare the physiological parameter(s) to a threshold(s) associated with the alarm 206, for example. In response to comparing the physiological parameter to the threshold(s), the processor(s) 112 may generate an alarm(s) of the customized set of alarms. For example, if the EtCO₂ parameter is 32 mmHg, the processor(s) 112 may determine that this EtCO₂ parameter is less than a threshold (e.g., the low limit of 33 mmHg) for the prioritized carbon dioxide alarm, and the processor(s) 112 may generate the carbon dioxide alarm in this scenario. The processor(s) 112 may also cause the alarm(s) to be output via the output device(s) 122, as described herein.

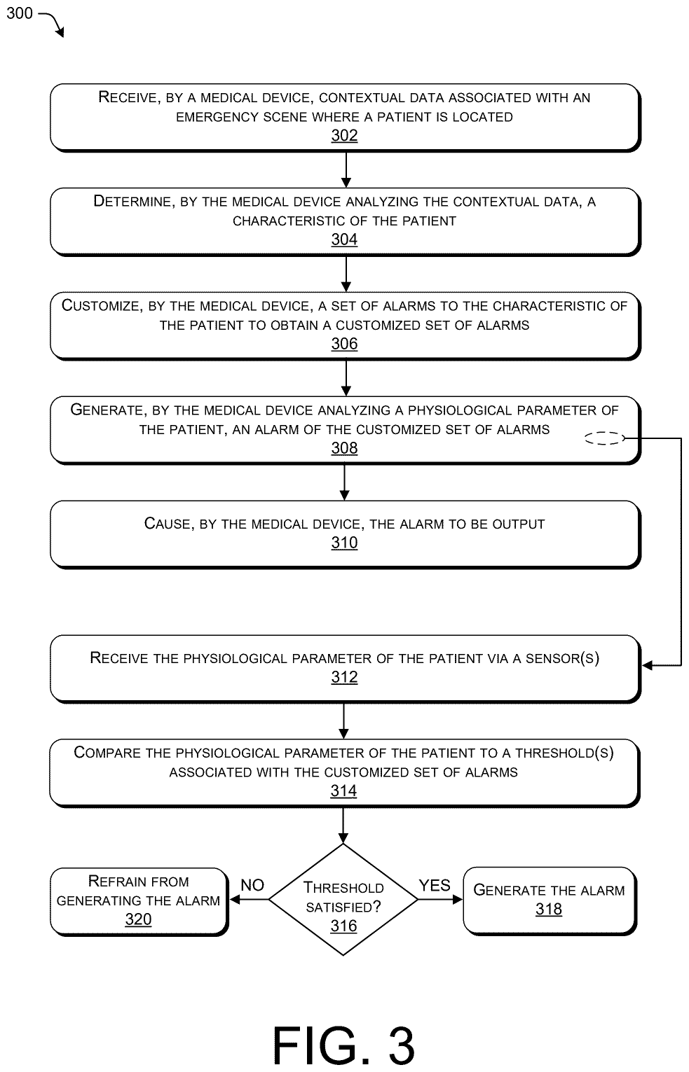

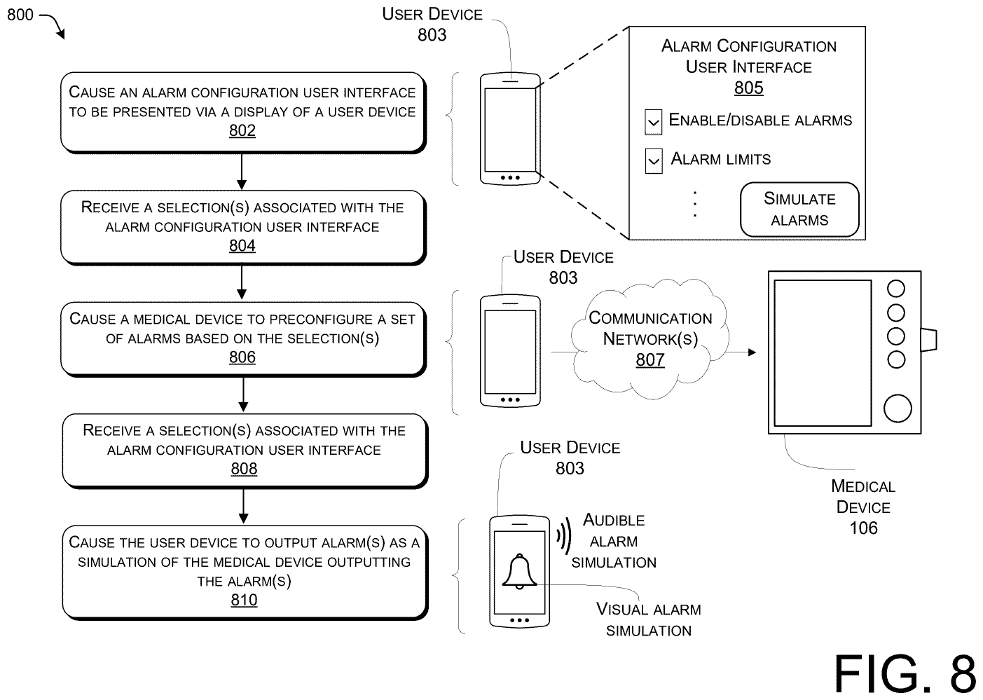

FIGS. 3, 4, 5, 6, 7, 8, and 9 illustrate processes and/or techniques performed by one or more systems, devices, or entities described herein. For example, the processes/techniques illustrated in FIGS. 3, 4, 5, 6, 7, and 9 may be implemented by the medical device 106, or at least one processor configured to execute instructions. As another example, the process illustrated in FIG. 8 may be implemented by a user device, or at least one processor configured to execute instructions. The processes described herein represent sequences of operations that can be implemented in hardware, software, or a combination thereof. In the context of software, the blocks represent computer-executable instructions stored on one or more computer-readable storage media that, when executed by a processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes. In some examples, an operation(s) of the process may be omitted entirely. Moreover, the processes described herein can be combined in whole or in part with each other or with other processes.

FIG. 3 illustrates an example process 300 for determining a characteristic of a patient from context of an emergency scene where the patient is located, and customizing a set of alarms of a medical device to the characteristic of the patient in order to output an alarm(s) that is customized to the patient. At 302, a medical device 106 receives contextual data 118 associated with an emergency scene 100A where a patient 104 is located. In some examples, an input device(s) 120 of the medical device 106 is configured to receive the contextual data 118 at 302. The input device(s) 120 that receives the contextual data 118 at 302 can include a microphone(s), a camera(s), a touch-sensitive display(s), a keypad(s), a cursor control device(s), a location-determining device(s) (e.g., a GPS receiver), a transceiver(s), or any combination thereof. The contextual data 118 received at 302 is indicative of a context of the emergency scene 100A and may include location data, audio data, image data (e.g., still images, video, etc.), environmental data, or any combination thereof.

At 304, the medical device 106 determines a characteristic of the patient 104 by analyzing the contextual data 118 received at 302. In some examples, a processor(s) 112 of the medical device 106 is configured to determine the characteristic of the patient 104 at 304 by executing the alarm customization module 116 and/or by processing the contextual data 118 or otherwise using the contextual data 118 to determine the characteristic of the patient 104. In some examples, analyzing the contextual data 118 at 304 includes providing the contextual data 118 as input to a trained artificial intelligence (AI) model(s) and receiving the characteristic(s) of the patient 104 as output from the trained AI model(s). The AI models described herein can be, or include, machine learning models. Machine learning generally involves processing a set of examples (called “training data” or a “training dataset”) in order to train the model(s). In some examples, the training dataset used to train the AI model(s) can include features and labels. However, the training dataset may be unlabeled, in some examples. Accordingly, the AI model(s) (e.g., machine learning model(s)) described herein may be trained using any suitable learning technique, such as supervised learning, unsupervised learning, semi-supervised learning, reinforcement learning, and so on. The training dataset can be represented by a set of features, such as in the form of an n-dimensional feature vector of quantifiable information about an attribute of the training dataset. In some examples, the training dataset may include sampled contextual data 118. Various features of the sampled contextual data 118 can be used to train the AI model(s) to output one or more characteristics of a patient. As the AI model(s) is/are trained, the AI model(s) learns how the features of the training dataset translate to the desired output (e.g., characteristic of a patient). For example, the AI model(s) can learn how the features of the contextual data 118 translate to patient characteristics. An AI model(s) (e.g., machine learning model(s)), once trained, is a learned mechanism that can receive new data as input and estimate or predict a result as output. For example, a trained machine learning model can comprise a classifier that is tasked with classifying unknown input (e.g., an unknown image) as one of multiple class labels (e.g., labeling the image as a cat or a dog). In some cases, a trained machine learning model is configured to implement a multi-label classification task (e.g., labeling images as “cat,” “dog,” “duck,” “penguin,” and so on). Additionally, or alternatively, a trained AI model can be trained to infer a probability, or a set of probabilities, for a classification task based on unknown data received as input. In some examples, the AI models described herein can be, or include, generative AI models, such as large language models (LLMs), neural networks (e.g., generative adversarial networks (GANs), and/or the like, which may be configured to generate text, images, and/or other media as output. In the context of the present disclosure, the trained AI model(s) may generate patient characteristics as text, images, and/or other media, which is usable to customize a set of alarms, as described herein.

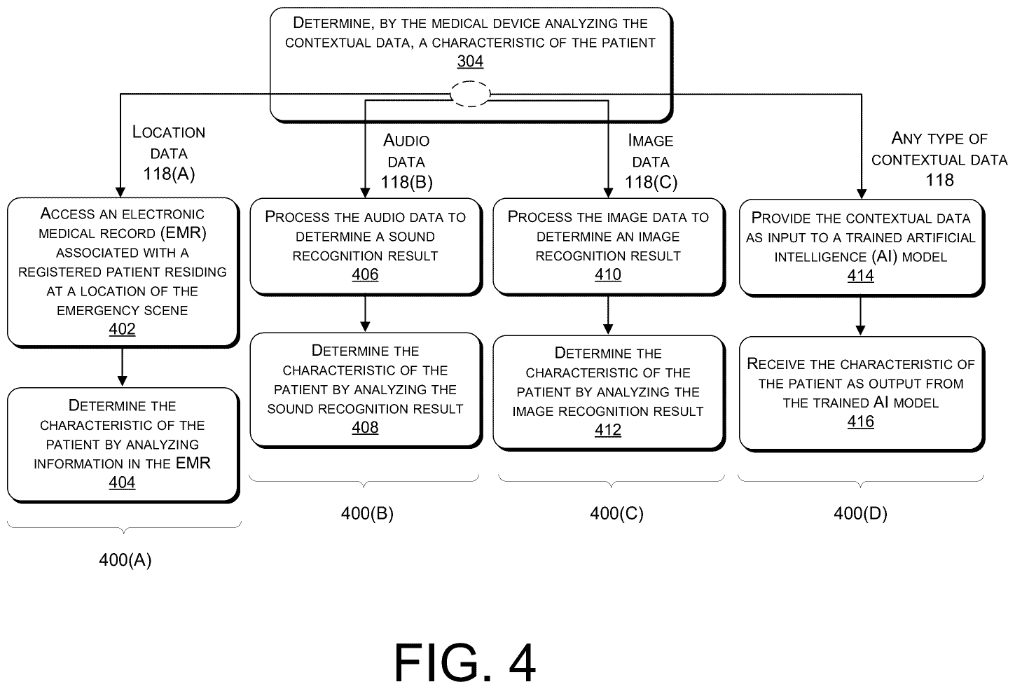

In some examples, analyzing the contextual data 118 at 304 includes accessing, from a datastore, historical data associated with characteristics of patients located at emergency scenes in the past, and analyzing a subset of the historical data based on the contextual data 118 to determine the characteristic(s) of the patient 104. In some examples, the characteristic(s) of the patient 104 is inferred from the contextual data 118 in the sense that the characteristic(s) of the patient 104 is deduced from the contextual data 118 as a likely characteristic of the patient 104. In some examples, the characteristic(s) of the patient 104 is a medical condition of the patient 104, such as a respiratory problem (e.g., asphyxiation, shortness of breath, etc.), a cardiovascular problem (e.g., a heart attack), and/or the like. In some examples, the characteristic(s) of the patient 104 is an attribute(s) or a classification(s) of the patient 104 as one of multiple class labels (e.g., obese or not obese, adult or youth/child, conscious or unconscious, breathing or not breathing, etc.), which can be used to infer a medical condition of the patient 104. Example techniques for determining the characteristic(s) of the patient 104 by analyzing different types of contextual data 118 are described in more detail elsewhere herein, including the description of FIG. 4, below.

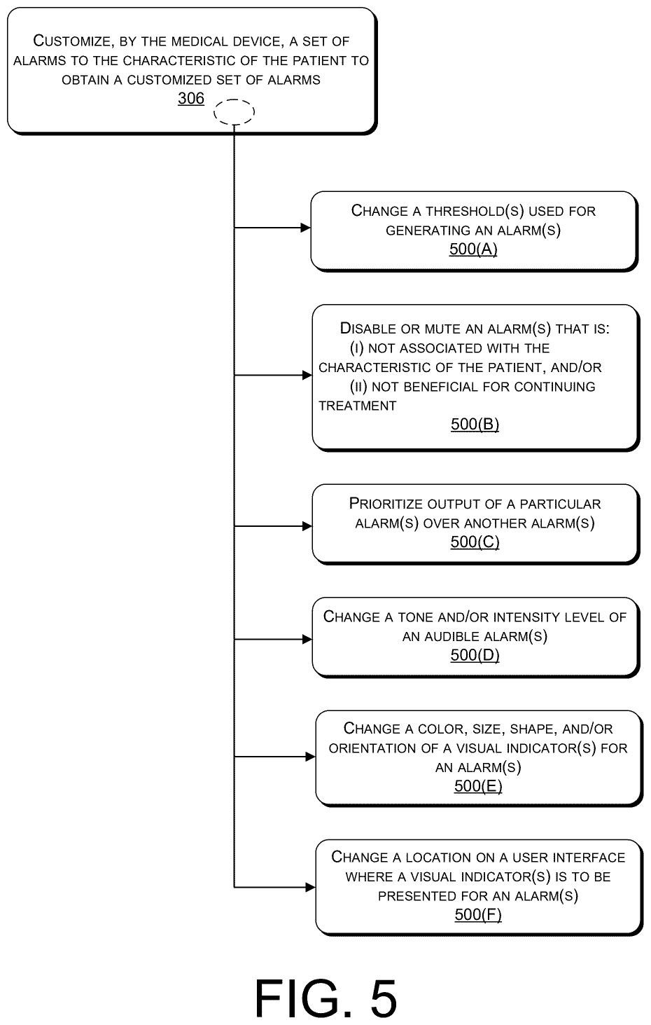

At 306, the medical device 106 customizes a set of alarms to the characteristic(s) of the patient 104 to obtain a customized set of alarms. In some examples, the processor(s) 112 of the medical device 106 is configured to customize the set of alarms at 306 by executing the alarm customization module 116 and/or by using the characteristic(s) of the patient 104 to lookup information on how to customize the set of alarms to the characteristic(s) of the patient 104. Example techniques for customizing a set of alarms are described in more detail elsewhere herein, including the description of FIG. 5, below. In general, the customized set of alarms obtained at 306 are tailored to the patient 104 and/or to a medical condition of the patient 104. To illustrate with one example, customizing the set of alarms at 306 may include changing a threshold(s) used for generating an alarm (e.g., changing the high and/or low limits associated with a particular alarm(s) to make the alarm(s) more or less sensitive and/or more appropriately tailored to the medical condition of the patient 104).

At 308, the medical device 106 generates an alarm(s) of the customized set of alarms by analyzing a physiological parameter(s) of the patient 104. In some examples, the processor(s) 112 of the medical device 106 is configured to generate the alarm(s) at 308 by processing the physiological parameter(s) of the patient 104 or otherwise using the physiological parameter(s) to determine whether to generate the alarm(s) or refrain from generating the alarm(s). In some examples, the alarm(s) generated at 308 is associated with a medical condition of the patient 104 determined at 304. An example technique for generating the alarm(s) at 308 will be discussed below with respect to sub-blocks 312-320.

At 310, the medical device 106 causes the alarm(s) to be output. In some examples, the processor(s) 112 of the medical device 106 is configured to cause the alarm(s) to be output at 310 via an output device(s) 122 of the medical device 106. The output device(s) 122 that outputs the alarm(s) at 310 can include a display(s), a light emitting element(s) (e.g., a LED(s)), an electrochromic material(s), a speaker(s), a haptic actuator(s), a transceiver(s), a printer(s), or any combination thereof. The alarm(s) output at 310 can be a visual alarm, an audible alarm, a tactile alarm, or any other suitable type of alarm that is designed to alert someone (e.g., a rescuer 102 at the emergency scene 100A) as to a medical condition of the patient 104. An example technique for generating the alarm(s) at 308 will now be described with respect to sub-blocks 312-320.

At 312, the medical device 106 (e.g., the processor(s) 112 thereof) receives the physiological parameter(s) of the patient 104 via a sensor(s) 108 of, or communicatively coupled to, the medical device 106. Examples of the sensor(s) 108 and the physiological parameter(s) the sensor(s) 108 is configured to detect are described above.

At 314, the medical device 106 (e.g., the processor(s) 112 thereof) compares the physiological parameter(s) of the patient 104 to a threshold(s) associated with the customized set of alarms. For example, the customized set of alarms may include one or more types of alarms, such as a heart rate alarm, a VF/ventricular tachycardia (VT) alarm, a carbon dioxide alarm, a pressure (e.g., arterial pressure, pulmonary artery pressure, etc.) alarm, and/or the like, and each alarm may be associated with one or more threshold(s) (e.g., high and/or low limits) that are used to generate the alarm(s). As noted above, one or more of these thresholds may have been changed in the customization of the set of alarms performed at 306. Accordingly, in some examples, the physiological parameter(s) of the patient 104 may be compared to a customized alarm limit(s) at 314, such as the customized alarm limit(s) 124 and/or the customized alarm limit(s) 134 described above with reference to FIGS. 1A and 1B, respectively.

At 316, a determination is made by the medical device 106 (e.g., the processor(s) 112 thereof) as to whether the threshold(s) is satisfied. As used herein, a value (e.g., a value of a physiological parameter) can “satisfy” a threshold if the value is: (i) equal to or greater than the threshold, (ii) strictly greater than the threshold, (iii) equal to or less than the threshold, or (iv) strictly less than the threshold. Whether an alarm is triggered by a physiological parameter being too high and/or too low may depend on the nature of the alarm. For example, a heart rate that is too high or too low may trigger a heart rate alarm. As such, the heart rate alarm can be triggered by either condition, whereas other alarms may only be triggered by one or the other condition. If, at 316, in response to comparing the physiological parameter(s) to the threshold(s), it is determined that the threshold is satisfied, the process 300 follows the YES route from 316 to 318 where the alarm(s) is generated. If, at 316, in response to comparing the physiological parameter(s) to the threshold(s), it is determined that the threshold is not satisfied, which may be the case when the physiological parameter(s) is within the alarm limits (e.g., high and low limits) associated with the alarm(s), the process 300 follows the NO route from 316 to 320 where the medical device 106 (e.g., the processor(s) 112 thereof) refrains from generating the alarm(s). In this scenario, blocks 308-310 would not be performed in the particular iteration of the process 300.

It is to be appreciated that other techniques for generating the alarm(s) at 308 may be implemented, in some examples. For example, generating the alarm(s) at 308 may include providing the physiological parameter(s) of the patient 104 as input to a trained AI model(s) (e.g., a machine learning model(s)) and receiving a classification (e.g., alarm triggered or alarm not triggered) as output from the trained AI model(s). Such an AI model(s) may be trained in similar ways to those described above. For example, an AI model(s) may be trained to learn how the features of the physiological parameters translate to a decision to generate, or not generate, an alarm(s).

FIG. 4 illustrates example techniques for determining a characteristic(s) of a patient from context of an emergency scene where the patient is located. As indicated in FIG. 4, the example techniques illustrated in FIG. 4 may be performed as sub-operations of block 304 of the process 300 described above. With reference to a first technique 400(A), the contextual data 118 includes location data 118(A) representing a location of the emergency scene 100A where the patient 104 is located. For example, the location data 118(A) may include GPS coordinates (e.g., latitude and longitude) received by a GPS receiver of the medical device 106, and, in some examples, the processor(s) 112 of the medical device 106 is configured to resolve the GPS coordinates to a residential address or a business address if the nearest address is within a threshold distance from the GPS coordinates. In some examples, the location data 118(A) is received via user input provided to the medical device 106 (e.g., via a touch-sensitive display, keypad, etc.). In this example, the rescuer 102 may enter an address or cross streets that correspond to the location of the emergency scene 100A upon arrival at the emergency scene 100A.

At 402, the medical device 106 accesses, from a datastore, an electronic medical record (EMR) associated with a registered patient residing at the location of the emergency scene 100A. For example, the medical device 106 may have access to a datastore containing EMRs of patients who have been registered at a medical care facility (e.g., a hospital, a clinic, a doctor's office, etc.), and the processor(s) 112 of the medical device 106 may use the location represented by the location data 118(A) to identify any EMRs associated with a registered patient residing at the location.

At 404, after an EMR associated with a registered patient residing at the location is identified and accessed at 402, the medical device 106 determines the characteristic(s) of the patient 104 at the emergency scene 100A by analyzing information in the EMR. For example, the EMR accessed at 402 may include information indicating that a registered patient residing at the location of the emergency scene 100A has a history of a medical condition (e.g., a history of heart attacks, respiratory problems, etc.). In these examples, based on a reasonable likelihood that the patient 104 located at the emergency scene 100A is the same person as the registered patient associated with the EMR, the processor(s) 112 of the medical device 106 may infer that the patient 104 at the emergency scene 100A is suffering from the medical condition indicated in the EMR (e.g., a heart attack, a respiratory problem, etc.).

With reference to a second technique 400(B), the contextual data 118 includes audio data 118(B) representing a sound(s) in an environment of the medical device 106 while the medical device 106 is located at the emergency scene 100A. For example, the medical device 106 may include an input device 120 in the form of a microphone(s) 200, and the contextual data 118, in this example, is in the form of audio data 118(B). In this example, the microphone(s) 200 may capture a sound(s) in an environment of the medical device 106 while the medical device 106 is located at an emergency scene 100A, and the audio data 118(B) may be generated based on the captured sound(s).

At 406, the processor(s) 112 of the medical device 106 processes the audio data 118(B) to determine a sound recognition result. Any suitable sound recognition technology can be used to process the audio data 118(B) to determine a sound recognition result. In some examples, event detection software can recognize sounds in the audio data 118(B), such as breathing sounds, crying sounds, the sound of glass breaking, and/or the like. In some examples, speech recognition software may be used to recognize speech in the audio data 118(B), which may involve the utilization of automatic speech recognition and/or natural language understanding algorithms and/or models. This might allow for the sound recognition result determined at 406 to indicate something important or relevant uttered by the rescuer 102 and/or the patient 104 (if the patient 104 is conscious and talking) at the emergency scene 100A. In some examples, the sound recognition result may indicate that the patient 104 is speaking, regardless of what the patient 104 is saying, which may suggest that the patient 104 is likely conscious. In some examples, the sound recognition result determined at 406 may be indicative of agonal breathing 202, which may suggest that the patient 104 is likely experiencing a respiratory problem.

At 408, the processor(s) 112 of the medical device 106 determines the characteristic(s) of the patient 104 at the emergency scene 100A by analyzing the sound recognition result. For example, if the sound recognition result is indicative of agonal breathing 202, the processor(s) 112 of the medical device 106 may determine, from this sound recognition result, that the patient 104 is likely experiencing a respiratory problem. In another example, the rescuer 102 may utter the phrase “we've got heart failure!”, and the processor(s) 112 may determine, from a sound recognition result indicative of this phrase, that the patient 104 is likely experiencing a heart attack.

With reference to a third technique 400(C), the contextual data 118 includes image data 118(C) representing at least a portion of the emergency scene 100A. For example, the medical device 106 may include an input device 120 in the form of an outward-facing camera(s), and the contextual data 118 received by this input device 120 is in the form of image data 118(C) (e.g., one or more still images, a video, etc.). In this example, the camera(s) of the medical device 106 may capture images of at least part of the emergency scene 100A that is within a field of view of the camera(s). In some examples, the patient 104 is within the field of view of the camera(s) when the image data 118(C) is generated such that the image data 118(C) represents the patient 104 at the emergency scene 100A. In some examples, the image data 118(C) represents the rescuer 102, bystanders, additional medical devices 110, a landscape, the sky, buildings, vehicles, or other structures and/or objects in the environment of the medical device 106, and/or the like. In some examples, the rescuer 102 may be wearing a headset and/or glasses with an outward-facing camera(s), and an input device 120 of the medical device 106 in the form of a transceiver(s) may receive the image data 118(C) from the headset and/or glasses worn by the rescuer 102.

At 410, the processor(s) 112 of the medical device 106 processes the image data 118(C) to determine an image recognition result. Any suitable image recognition technology can be used to process the image data 118(C) to determine an image recognition result. In some examples, the image data 118(C) is provided as input to a trained AI model(s) and the image recognition result is received as output from the trained AI model(s). Such an AI model(s) may be trained in similar ways to those described above. For example, an AI model(s) may be trained to learn how the features of the image data 118(C) translate to particular image recognition results.

At 412, the processor(s) 112 of the medical device 106 determines the characteristic(s) of the patient 104 by analyzing the image recognition result. In some examples, the image recognition result is indicative of a size of the patient 104, and the characteristic(s) of the patient 104 determined at 412 may be that the patient 104 is obese, an adult, a youth/child, an infant, and/or the like, which may be useful for inferring a medical condition of the patient 104. In some examples, the characteristic(s) of the patient 104 determined at 412 may indicate a gender of the patient 104, an age (e.g., young, middle aged, or old) of the patent 104, which may be useful for inferring a medical condition of the patient 104. In some examples, the characteristic(s) of the patient 104 determined at 412 may be that the patient 104 is conscious or unconscious, bleeding or not bleeding, vomiting or not vomiting, and/or the like. In some examples, the image recognition result may indicate black or dense smoke at the emergency scene 100A (e.g., from a fire), and the characteristic(s) of the patient 104 determined at 412 may be that the patient 104 is likely suffering from burns and/or a respiratory problem (e.g., smoke inhalation).

With reference to a fourth technique 400(D), the contextual data 118 may be any suitable type of data, such as the above-described location data 118(A), audio data 118(B), image data 118(C), and/or other types of data including, without limitation, environmental data representing a condition of an environment of the emergency scene 100A (e.g., an environmental condition associated with air temperature, air quality, and/or flooding in the environment), physiological data representing a physiological parameter of the patient 104, and/or the like. In some examples, environmental data is received from one or more server(s) over a network(s) (e.g., the Internet), such as a server(s) associated with a weather and/or news reporting service.

At 414, the processor(s) 112 of the medical device 106 provides the contextual data 118 as input to a trained AI model(s) (e.g., a machine learning model(s)), and at 416, the processor(s) 112 receives the characteristic(s) of the patient 104 as output from the trained AI model(s). For instance, the trained AI model(s) may be configured to analyze the contextual data 118 to predict a characteristic(s) of the patient 104, such as a medical condition of the patient. This could be based on processing location, audio, images, and/or any other type of contextual data 118. In some examples, whether or not a trained AI model(s) is utilized to determine the characteristic(s) of the patient 104, if the contextual data 118 includes environmental data representing a condition of an environment of the emergency scene 100A, such as an air temperature above a threshold temperature, the characteristic(s) of the patient 104 may be that the patient 104 is likely experiencing heat stroke due to a heat wave in the vicinity of the emergency scene 100A. In another example, the environmental data may indicate that an air quality index (AQI) in the environment of the emergency scene 100A has fallen below a threshold AQI, and the characteristic(s) of the patient 104 may be that the patient 104 is likely experiencing a respiratory problem due to the smoke from a nearby wildfire that has caused the decrease in AQI, for example. In yet another example, if the contextual data 118 includes physiological data representing a physiological parameter and/or an attribute of the patient 104 (e.g., that the patient 104 is obese), the characteristic(s) of the patient 104 may be that the patient 104 is likely experiencing a respiratory problem or a cardiovascular problem. In these examples, the size of the patient 104 can be inferred from image data 118(C) and/or data from a nearby medical device 110, such as a mechanical chest compression device configured to measure a size of the patient 104 via an amount of adjustment of the device in order to fit the device around the torso of the patient 104, an amount of pressure detected by pressure sensors in the backplate of the device, and/or the like. In some examples, a cot or a stretcher may have pressure sensors that can detect an amount of pressure indicative of a size of the patient 104, which may be received by a transceiver(s) of the medical device 106 and used to infer that the patient 104 is obese.

FIG. 5 illustrates example techniques for customizing a set of alarms. As indicated in FIG. 5, the example techniques illustrated in FIG. 5 may be performed as sub-operations of block 306 of the process 300 described above. However, it is to be appreciated that the techniques illustrated in FIG. 5 may be used to customize a set of alarms in any of the contexts and/or scenarios described herein.

With reference to a first technique 500(A), customizing a set of alarms of the medical device 106 may include changing a threshold(s) used for generating an alarm(s). For example, the limit(s) (e.g., the high limit and/or low limit) associated with a particular alarm(s) of the set of alarms for the medical device 106 may be changed from a wider range to a narrower range, or vice versa, in order to adjust the sensitivity of the alarm, and/or the range between the high and low limit may be changed to a different range that is tailored to the characteristic(s) (e.g., medical condition) of the patient 104. As an illustrative example, a ST-segment elevation myocardial infarction (STEMI) detection threshold(s) can be changed so that it is tailored to a patient 104 who has a history of heart attacks (e.g., the history of heart attacks having been determined from information in an EMR associated with the patient 104, from device data 132 received from a patient device 130, etc.). As another example, the limits associated with a heart rate alarm may be set to a relatively wide range (e.g., a high limit of 110 bpm and a low limit of 45 bpm) by default, and customizing a set of alarms, including the heart rate alarm, may involve changing the heart rate alarm limit(s) to a narrower range of, say, a high limit of 100 bpm and a low limit of 50 bpm. For instance, if the characteristic(s) of the patient 104 is that the patient 104 is likely experiencing a heart attack, the customized heart rate alarm limits may allow for monitoring the patient's 104 heart rate closer to a normal heart rate (e.g., 72 bpm), or closer to a previously recorded heart rate of the patient 104.

With reference to a second technique 500(B), customizing a set of alarms of the medical device 106 may include disabling or muting one or more alarms of the set of alarms for the medical device 106, such as alarms that are (i) not associated with a determined characteristic(s) (e.g., medical condition) of the patient 104, or (ii) associated with the characteristic(s) (e.g., medical condition), but not beneficial for continuing treatment. For example, if the characteristic(s) of the patient 104 is that the patient 104 is likely experiencing a heart attack, one or more alarms that are not associated with a cardiovascular-related medical condition can be disabled or muted, and/or cardiovascular-related alarms that are not beneficial for continuing treatment of the heart attack may be disabled or muted.

With reference to a third technique 500(C), customizing a set of alarms of the medical device 106 may include prioritizing output of a particular alarm(s) over one or more additional alarms of the set of alarms. For example, if a respiratory-related alarm is prioritized over a cardiovascular-related alarm, in a scenario where both alarms are triggered at or near the same time, the processor(s) 112 of the medical device 106 may generate the respiratory-related alarm and cause the respiratory-related alarm to be output while refraining from generating the cardiovascular alarm, or suppressing the cardiovascular alarm in some way, such as disabling an audible alarm for the cardiovascular-related alarm and strictly outputting, on the display(s) of the medical device 106, a visual alarm for the cardiovascular-related alarm.

With reference to a fourth technique 500(D), customizing a set of alarms of the medical device 106 may include changing a tone and/or an intensity level associated with an alarm(s) of the set of alarms. For example, a tone of a prioritized alarm(s) may be changed to a tone that is more conspicuous and/or noticeable to a rescuer 102 at the emergency scene 100A where the patient 104 is located, and a tone of a deprioritized alarm(s) may be changed to a tone that is more subtle or inconspicuous so that sensory overload of the rescuer 102 is mitigated. As another example, an intensity level at which a prioritized alarm(s) is to be output via the output device(s) 122 may be increased to make the prioritized alarm(s) more conspicuous, while an intensity level at which a deprioritized alarm(s) is to be output via the output device(s) 122 may be decreased to make the deprioritized alarm(s) more inconspicuous.

With reference to a fifth technique 500(E), customizing a set of alarms of the medical device 106 may include changing a color, a size, a shape, and/or an orientation of a visual indicator that is to be presented via a user interface on the display(s) of the medical device 106. For example, a visual indicator for a prioritized alarm(s) may be changed to a red color and/or enlarged so that it is more conspicuous, while a visual indicator for a deprioritized alarm(s) may be changed to a more subtle color (e.g., amber, orange, etc.) and/or reduced in size so that it is more inconspicuous.

With reference to a sixth technique 500(F), customizing a set of alarms of the medical device 106 may include changing a location on the user interface where the visual indicator is to be presented. For example, a visual indicator associated with a prioritized alarm(s) may be presented at or near a center of the display to make it more conspicuous, and a visual indicator associated with a deprioritized alarm(s) may be presented at or near a periphery of the display to make it more inconspicuous. In some examples, the customizing at 500(F) may involve selecting a display amongst multiple displays for outputting a visual indicator associated with the alarm(s). For example, if the rescuer 102 is wearing a head-mounted display, the customizing at 500(F) may involve selecting the head-mounted display for output of the alarm(s) so that the rescuer 102 is more likely to notice the visual indicator. As another example, the patient 104 may be wearing a patient device 130, such as a smart watch, and the customizing at 500(F) may involve selecting the patient device 130 for output of the alarm(s) so that the patient 104 is more likely to notice the visual indicator.

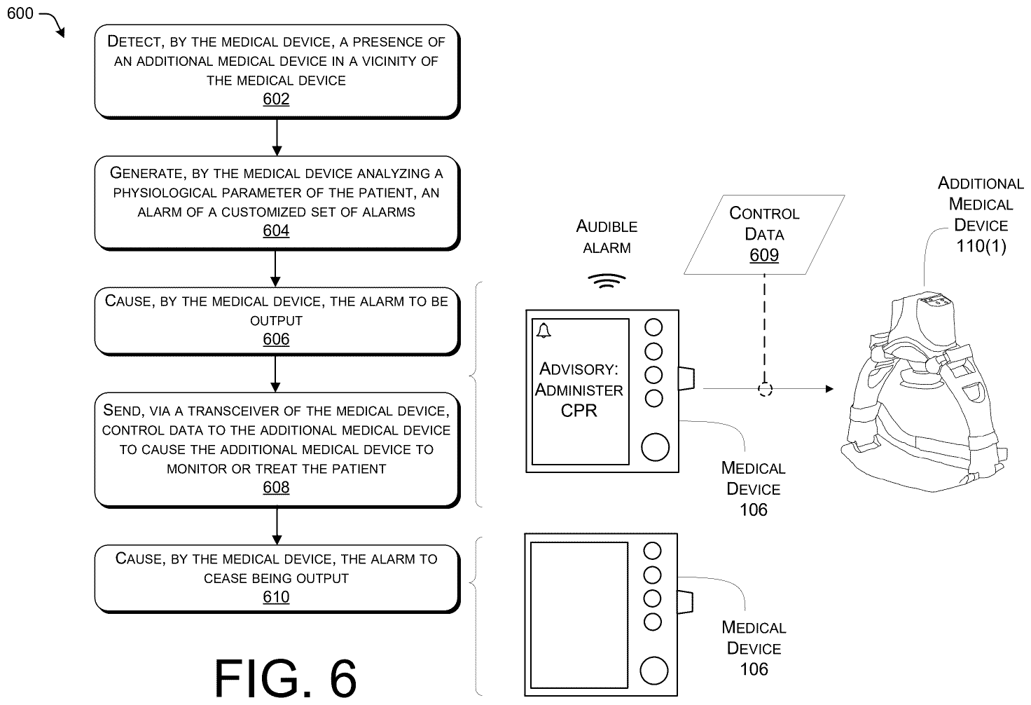

Conventional medical device alarms are output without consideration of the availability of additional medical devices to monitor and/or treat a patient. Moreover, conventional medical devise do not take any action automatically to overcome an alarm condition. FIG. 6 illustrates an example process 600 for managing alarms of a medical device in the context of one or more additional medical devices in a vicinity of the medical device.

At 602, a medical device 106 detects a presence of an additional medical device 110(1) in a vicinity of the medical device 106. The detection at 602 can be implemented in various ways, such as by using an outward-facing camera(s) of the medical device 106 to capture one or more images (e.g., still images, video, etc.) of the additional medical device 110(1) and processing the resulting image data to detect the additional medical device 110(1) from the captured image(s). As another example, as described above, a pairing procedure (e.g., based on a wireless protocol, such as BLUETOOTH, a manual pairing process, such as a tap-to-pair process, etc.) may be utilized between the medical device 106 and the additional medical device 110(1) at an emergency scene 100A, and the detection at 602 may be based at least in part on such a pairing procedure.

At 604, the medical device 106 generates an alarm(s) of a customized set of alarms by analyzing a physiological parameter(s) of the patient 104. In some examples, the processor(s) 112 of the medical device 106 is configured to generate the alarm(s) at 604 by processing the physiological parameter(s) of the patient 104 or otherwise using the physiological parameter(s) to determine whether to generate the alarm(s) or refrain from generating the alarm(s). In some examples, the alarm(s) generated at 604 is associated with a medical condition of the patient 104, such as a heart-related medical condition, a respiratory-related medical condition, etc.

At 606, the medical device 106 causes the alarm(s) to be output. In some examples, the processor(s) 112 of the medical device 106 is configured to cause the alarm(s) to be output at 606 via an output device(s) 122 of the medical device 106. The alarm(s) output at 606 can be a visual alarm, an audible alarm, a tactile alarm, or any other suitable type of alarm that is designed to alert someone (e.g., a rescuer 102) as to a medical condition of the patient 104. In an illustrative example, the alarm(s) output at 606 can be a VF/VT alarm.

At 608, in response to the causing the alarm(s) to be output at 606, the medical device 106 sends, via a transceiver(s) 128 of the medical device 106, control data 609 to the additional medical device 110(1) to cause the additional medical device 110(1) to monitor or treat the patient 104. The sending of the control data 609 at 608 may be based on a determination that the alarm(s) being output is associated with the additional medical device(s) 110(1) and/or with a treatment that the additional medical device 110(1) is configured to administer to the patient 104. In the example of FIG. 6, the control data 609 may instruct the additional medical device 110(1) to begin chest compressions, since the additional medical device 110(1) depicted in FIG. 6 is a mechanical chest compression device. However, other types of additional medical devices 110 can be controlled at 608. For example, the control data 609 may activate an infusion pump with preloaded medication if the alarm(s) being output at 606 is generated in response to a blood pressure of the patient 104 satisfying a threshold(s) (e.g., exceeding a high limit of a blood pressure alarm). In this manner, the patient 104 can be treated automatically via an additional medical device 110 that is preauthorized by a caretaker (e.g., a nurse) to treat the patient 104 without human intervention.

At 610, in response to the sending the control data 609 to the additional medical device 110(1), the medical device 106 may cause the alarm(s) to cease being output. For example, if the alarm(s) is designed to alert the rescuer 102 that the patient 104 is in need of cardiopulmonary resuscitation (CPR) treatment, and if the medical device 106 automatically controls the additional medical device 110(1) to commence CPR, chest compressions, and/or ventilation at 608, then it may be unnecessary to continue outputting the alarm(s), since the additional medical device 110(1) has started to address the alarm condition by commencing treatment that is related to the alarm(s).

Other ways of suppressing alarms to mitigate alarm fatigue are contemplated herein, such as allowing a rescuer to issue a voice command to stop an ongoing alarm, and if such a voice command is detected via a microphone(s) 200 of the medical device 106 and using speech processing software to recognize the intent of the voice command, the medical device 106 may cause the ongoing alarm to cease being output. This can allow a user (e.g., the rescuer 102) to quickly shut down an alarm(s) on-demand. Additionally, or alternatively, one or more interactive elements may be presented on a touch-sensitive display of the medical device 106, and the rescuer 102 may select an interactive element associated with an ongoing alarm to disable the alarm, thereby mitigating alarm fatigue through a simple interaction with a touch-sensitive display.

In some examples, the medical device 106 may be configured to notify personnel qualified to address an alarm in response to generating and/or outputting the alarm. For example, the medical device 106 may be configured to send an electronic mail (email) message, a text message, a push notification, and/or the like to qualified personnel, such as an additional rescuer or rescue team in transit to the emergency scene where the patient 104 is located. To illustrate, if a low battery alarm(s) is generated and output by the medical device 106, a notification may be sent automatically by the medical device 106 to a battery replacement team that is responsible for addressing that type of alarm(s). In some examples, the medical device 106 may be configured to notify a remote system (e.g., a centralized alarm hub) about a generated alarm, wherein the remote system is configured to monitor deployed medical devices including the medical device 106. In this manner, the remote system can be made aware of any medical device in the field that has generated an alarm while monitoring the state of the devices in the field.

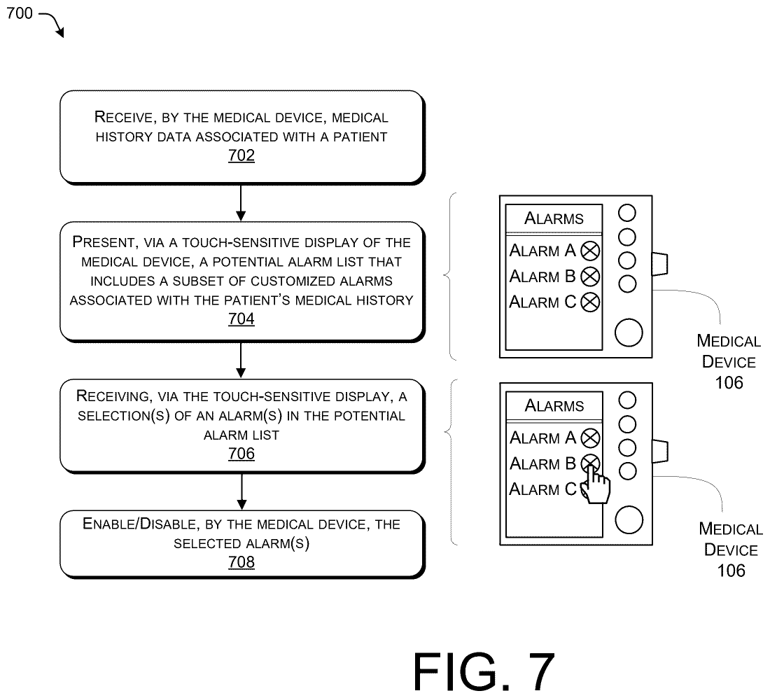

Conventional medical device alarms are output without consideration of the medication administered to a patient and/or the medical history of the patient. FIG. 7 illustrates an example process 700 for managing alarms of a medical device based on a medical history of a patient at an emergency scene.

At 702, a medical device 106 receives medical history data associated with a patient 104 at an emergency scene 100A. The medical history data may be obtained from information in an EMR of the patient 104 that is accessible to the medical device 106 (e.g., via a network(s)) based on identifying the patient 104 and/or after determining a location of the emergency scene 100A and looking up an EMR of a registered patient who resides at the location. In some examples, the medical history data indicates a medication administered to the patient 104 in the past, a medical condition of the patient 104 that occurred in the past, and/or the like. In some examples, the medical history data includes medication data representing a medication administered to a patient 104 at an emergency scene 100A. In some examples, the medical device 106 receives such medication data via user input to the medical device 106 (e.g., the rescuer 102 may enter the medication data via a touch-sensitive display of the medical device 106, a keypad of the medical device 106, and/or the like). In some examples, the medication data is received from an additional medical device 110(2) (e.g., a medication-administering device, such as an IV pump) via a transceiver(s) 128 of the medical device 106. This may occur after the medical device 106 and the additional medical device 110(2) are paired at the emergency scene 110A, as described above.

At 704, the medical device 106 presents, via a touch-sensitive display of the medical device 106, a potential alarm list that includes a subset of a customized set of alarms, wherein the subset is associated with a medical history of the patient 104 determined from the medical history data. In some examples, the alarm(s) in the potential alarm list is associated with the medication administered to the patient 104 at the emergency scene 100A.

At 706, the medical device 106 receives, via the touch-sensitive display, one or more selections of one or more alarms in the potential alarm list. For example, as illustrated in FIG. 7, an operator of the medical device 106 (e.g., the rescuer 102) may select an interactive element associated with “Alarm B” in the potential alarm list.

At 708, in response to the receiving of the one or more selections of the one or more alarms, the medical device 106 enables or disables the one or more alarms. For example, “Alarm B” may be disabled if the operator (e.g., the rescuer 102) selects “Alarm B” in the potential alarm list and/or an icon that is indicative of disabling the alarm (e.g., an “x” icon, an “off” icon, etc.). As another example, an alarm in the potential alarm list may be enabled if the operator (e.g., the rescuer 102) selects the alarm in the potential alarm list and/or an icon that is indicative of enabling the alarm (e.g., a “check mark” icon, an “on” icon, etc.). Accordingly, the process 700 can allow an operator of the medical device 106, such as the rescuer 102, to manage the alarms in a way that mitigates alarm fatigue by enabling and/or disabling certain alarms, such as by disabling certain alarms the operator does not deem beneficial for monitoring and/or treating the patient 104 at the emergency scene 100A. The potential alarm list may be displayed during initial set-up of an alarm, in some examples, and may be accessed by a user (e.g., a rescuer 102, a nurse, etc.) at any time. This helps users to prevent the alarm by taking appropriate action.

Conventional alarm preset offerings can lead to excessive alarms output by a medical device, as there is typically a single default preset option for all patient conditions. Some conventional alarm preset offerings allow a user to select basic options (e.g., Wide versus Narrow, single-digit adjustment of numerical values for physiological parameters of interest, etc.). Furthermore, protocols of emergency medical services (EMS) teams and/or medical directors at a hospital, clinic, and/or the like are not incorporated into conventional alarm preset offerings. For example, EMS directors typically have specific protocols for dealing with particular scenarios that arise during a resuscitation attempt of a patient. These are often based on the patient's condition and, at times, are based on changes in physiological parameters that can potentially go unnoticed at the scene despite monitoring. Some of the treatment protocols are standard across a country or region but some of these are unique to a director's service. As such, the burden is placed on EMS crews to notice changes in patient parameters and to react to them immediately in accordance with their procedures entirely from the memory of the EMS crew personnel (e.g., trying to recall perfectly and instantly what the next treatment steps should be). FIG. 8 illustrates an example process 800 for preconfiguring the alarms of a medical device and simulating the alarms via an alarm configuration user interface presented on a user device.

At 802, a processor(s) of a user device 803 may cause an alarm configuration user interface 805 to be presented via a display of the user device 803. The alarm configuration user interface 805 allows a user (e.g., a caregiver, paramedic, etc.) to preconfigure a set of alarms that a medical device 106 is configured to output at emergency scenes. In some examples, the alarm configuration user interface 805 allows a user to input customized alarm settings that customize the set of alarms for the medical device 106 to various patient conditions (e.g., drowning, overdose, cardiac-related conditions, etc.), and/or to various protocols of EMS crews, medical directors, and/or the like. In some examples, the alarm configuration user interface 805 allows a user to access custom-made templates and/or alarm settings for download in order to preconfigure the set of alarms for the medical device 106 so that the alarms are relevant to a particular protocol of the EMS crew, medical director, etc. and/or to a common patient scope. In some examples, the alarm configuration user interface 805 allows a user to preconfigure specific notifications for advising treatments during use of the medical device 106, thereby making alerts and the circumstances in which they are triggered configurable by the user to create a flexible, user-centered notification system that aids in compliance with a specific treatment protocol. Configurable aspects of the alarms via the alarm configuration user interface 805 may include: (i) triggers (e.g., % or absolute values, and/or changes in selected physiological parameters such as blood pressure, EtCO₂, heart rate, etc.), (ii) messaging (e.g., specific wording, text size, alarm tones that are most effective for a given rescuer team, etc.), and/or (iii) treatment recommendations (e.g., what the rescuer 102 should do as a result of the given treatment. Consider an example where a medical director creates the following criteria: (a) rescue team diagnoses an inferior STEMI, (b) ST elevation in V1 and ST depression in V2 is detected, (c) non-invasive blood pressure (NIBP) shows patient is hypotensive. In this example, the alarm configuration user interface 805 may allow a user to create treatment considerations, recommendations, and/or reminders to activate a cath lab, treat with fluid bolus of xxx mL/kg, and/or avoid nitrates.

At 804, the user device 803 receives one or more first selections associated with the alarm configuration user interface 805. For example, the user of the user device 803 may interact with interactive elements (e.g., drop down menus, radio buttons, etc.) presented via the alarm configuration user interface 805 to enable or disable certain alarms, adjust the alarm limits associated with certain alarms, etc.

At 806, the user device 803 causes the medical device 106 to preconfigure the set of alarms based on the one or more first selections received at 804. For example, the user device 803 may send data to the medical device 106 via a communication network(s) 807 for preconfiguring the set of alarms based on the one or more first selections received at 804. As noted above, this may allow for customizing a set of alarms for the medical device 106 to various patient conditions and/or protocols of a EMS crew, medical director, etc.

At 808, the user device 803 receives one or more second selections associated with the alarm configuration user interface 805. For example, the one or more second selections may involve selecting an interactive element(s) (e.g., a “simulate alarms” button) to simulate one or more of the alarms of the set of alarms for the medical device 106.

At 810, a processor(s) of the user device 803 causes, based on the one or more second selections received at 808, the user device 803 to output the set of alarms in accordance with the one or more second selections as a simulation of the medical device 106 outputting the set of alarms. For example, the user device 803 may output audible alarms via a speaker(s) of the user device 803 as they would sound if the audible alarms were output via a speaker(s) of the medical device 106, and/or the user device 803 may present visual alarms via a display(s) of the user device 803 as they would look if the visual alarms were presented via a display(s) of the medical device 106. In this way, a user of the user device 803 can preconfigure alarms and simulate the alarms on the user device 803 to ensure that the alarms are preconfigured how the user wants them to be. That is, the user can see and hear the preconfigured and/or customized alarms to visualize how they would look and/or sound before they are deployed in the field. In some examples, an AI model(s) learns or is trained to recognize how, or in what ways, certain caregivers or paramedics could improve their treatment (such as the quality of ventilation), and the AI model(s) may suggest a customized set of alarms via the alarm configuration user interface 805 based on the improvement suggestions for the user group.

Accordingly, the process 800 and/or the alarm configuration user interface 805 can reduce the occurrence of alarm fatigue to improve patient outcomes/care by ensuring that alarms are tailored to specific patient scenarios and to reserve the generation and output of alarms for scenarios of interest to a rescuer(s) 102. This customization decreases cognitive load for rescuers during use of the medical device 106 while adapting to specific protocols of EMS crews, medical directors, etc. The process 800 and/or the alarm configuration user interface 805 can also assist in the sharing of best practices amongst different EMS crews, medical teams, etc. The alarm configuration user interface 805 is an effective tool that aids EMS crews to take specific actions established by their medical director, and the settings input via the alarm configuration user interface 805 may allow for a better understanding of how medical devices are used in the field and what aspects of patient conditions are most important to EMS crews, medical directors, etc., and/or how frequently certain triggers occur, which, in turn, could lead to the generation of new technologies and medical device features. This flexible, user-centric notification system also aids in compliance with specific treatment protocols whilst taking the burden of monitoring and/or recalling treatment steps away from the rescuer(s) 102 during what can often be a stressful and complicated environment at an emergency scene.

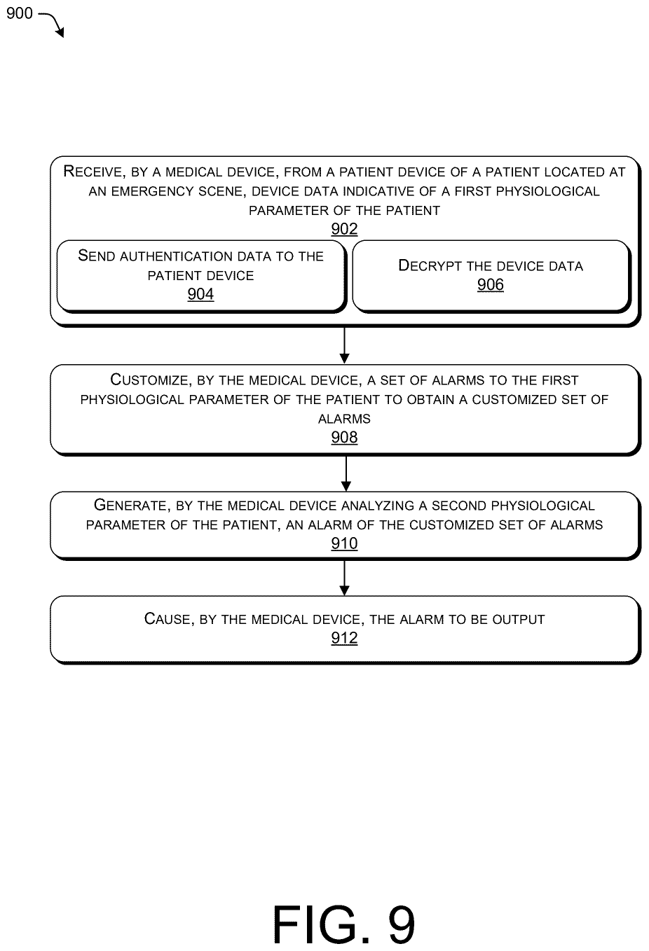

FIG. 9 illustrates an example process 900 for determining a physiological parameter of a patient from device data received by a medical device from a patient device of the patient, and customizing a set of alarms of the medical device to the physiological parameter of the patient in order to output an alarm(s) that is customized to the patient. At 902, a medical device 106 receives, from a patient device 130 of a patient 104 located at an emergency scene 100B, device data 132 indicative of a first physiological parameter(s) of the patient 104. In some examples, a transceiver(s) 128 of the medical device 106 is configured to receive the first physiological parameter(s) at 902. In some examples, a communication signal is received by the transceiver(s) 128, the communication signal including the device data 132 indicating the first physiological parameter(s) of the patient 104. In some examples, the device data 132 (and/or the communication signal including the device data 132) is received wirelessly using a wireless communication protocol (e.g., a short-range wireless protocol, such as BLUETOOTH, NFC, or the like). In some examples, a sensor 108 associated with the medical device 106 is configured to detect the first physiological parameter(s) of the patient 104. The first physiological parameter(s) can be any of the example physiological parameters described above, such as a heart rate, body temperature, blood pressure, blood oxygenation, and/or the like. In some examples, the patient device 130 is a wearable device worn by the patient 104, such as a smart watch. In some examples, the patient device 130 is an implantable device. In some examples, the patient device 130 is an implantable device other than a pacemaker. In some examples, the patient device 130 is a home health care device, such as an insulin pump. If the patient device 130 is owned by, and/or the property of, a hospital or a clinic, sharing data between the medical device 106 and the patient device 130 may involve proprietary protocols and/or algorithms that provide enhanced security from cyberattacks and/or enhanced protection/privacy of sensitive patient information.

At 904, in some examples, the medical device 106 sends, to the patient device 130, authentication data for authenticating the medical device 106 prior to receiving the device data 132 at 902. In some examples, the transceiver(s) 128 of the medical device 106 is configured to send the authentication data at 904. This may result in completing a “handshake” between the medical device 106 and the patient device 130 so that the patient device 130 can trust the medical device 106 with the device data 132 and/or so that the patient device 130 ensures that it is sending the device data 132 to a medical device associated with a care provider in need of any and all patient information in order to monitor and/or treat the patient 104. The sending of the authentication data at 904 can involve any of the authentication techniques described above.

At 906, in some examples, the device data 132 received at 902 is encrypted, and/or the device data 132 includes encrypted data indicating the first physiological parameter(s) of the patent 104, and the processor(s) 112 of the medical device 106 decrypts the device data 132 and/or the encrypted data. The decryption at 906 can involve any of the cryptographic techniques described above. The use of cryptographic techniques to send encrypted data between the medical device 106 and the patient device 130 helps to prevent sensitive patient information from being disclosed to and/or discoverable by unauthorized third parties.

At 908, the medical device 106 customizes a set of alarms to the first physiological parameter(s) of the patient 104 to obtain a customized set of alarms. In some examples, the processor(s) 112 of the medical device 106 is configured to customize the set of alarms at 908 by executing the alarm customization module 116 and/or by using the first physiological parameter(s) of the patient 104 to lookup information on how to customize the set of alarms to the characteristic(s) of the patient 104. Example techniques for customizing a set of alarms are described in more detail elsewhere herein, including the description of FIG. 5, above. In general, the customized set of alarms obtained at 908 are tailored to the patient 104 and/or the first physiological parameter(s) of the patient 104 and/or a medical condition of the patient 104. In some examples, a medical condition of the patient 104 is determined from the first physiological parameter(s), such as a determination that the patient 104 has had an arrhythmia for a period of time, and/or that the body temperature of the patient 104 has been above a threshold for a period of time (e.g., the patient 104 has had a fever for several hours). In these examples, the set of alarms may be customized to the determined medical condition at 908. Accordingly, because the device data 132 can provide valuable insights into a period of time prior to arrival of a rescuer 102 at the emergency scene 100B, the set of alarm(s) can be customized in ways that may not be achievable without having received the device data 132 from the patient device 130. In one example, customizing the set of alarms at 908 may include changing a threshold(s) used for generating an alarm (e.g., changing the high and/or low limits associated with a particular alarm(s) to make the alarm(s) more or less sensitive and/or more appropriately tailored to the medical condition of the patient 104).

At 910, the medical device 106 generates an alarm(s) of the customized set of alarms by analyzing a second physiological parameter(s) of the patient 104. In some examples, the processor(s) 112 of the medical device 106 is configured to generate the alarm(s) at 910 by processing the second physiological parameter(s) of the patient 104 or otherwise using the second physiological parameter(s) to determine whether to generate the alarm(s) or refrain from generating the alarm(s). In some examples, the alarm(s) generated at 910 is associated with a medical condition of the patient 104. Generating the alarm(s) at 910 may involve the example technique discussed above with respect to sub-blocks 312-320 of the process 300, and/or any other suitable technique described herein, such as using a trained AI model(s) to generate the alarm.

At 912, the medical device 106 causes the alarm(s) to be output. In some examples, the processor(s) 112 of the medical device 106 is configured to cause the alarm(s) to be output at 912 via an output device(s) 122 of the medical device 106. The output device(s) 122 that outputs the alarm(s) at 912 can include a display(s), a light emitting element(s) (e.g., a LED(s)), an electrochromic material(s), a speaker(s), a haptic actuator(s), a transceiver(s), a printer(s), or any combination thereof. The alarm(s) output at 912 can be a visual alarm, an audible alarm, a tactile alarm, or any other suitable type of alarm that is designed to alert someone (e.g., a rescuer 102) as to a medical condition of the patient 104.

In implementing the process 900, consider a scenario where a patient 104 is collapsed (e.g., unconscious) upon arrival of a rescuer 102 (e.g., a rescue team including the rescuer 102) at the emergency scene 100B, and there is an insulin pump (which is an example of a patient device 130) on or near the patient 104. The insulin pump sends device data 132 wirelessly to the medical device 106 (e.g., a monitor-defibrillator), and the medical device 106 receives the device data 132 at 902 and the process 900 is implemented. In this implementation of the process 900, the processor(s) 112 of the medical device 106 may determine that that the insulin pump has been malfunctioning for the past day, and infers that the patient 104 has a diabetes-related medical condition and/or an insulin-related medical condition. Based on this inference, the processor(s) 112 prioritizes alarms associated with diabetes and/or insulin-related medical conditions so that the alarms are tailored to the patient's medical condition.

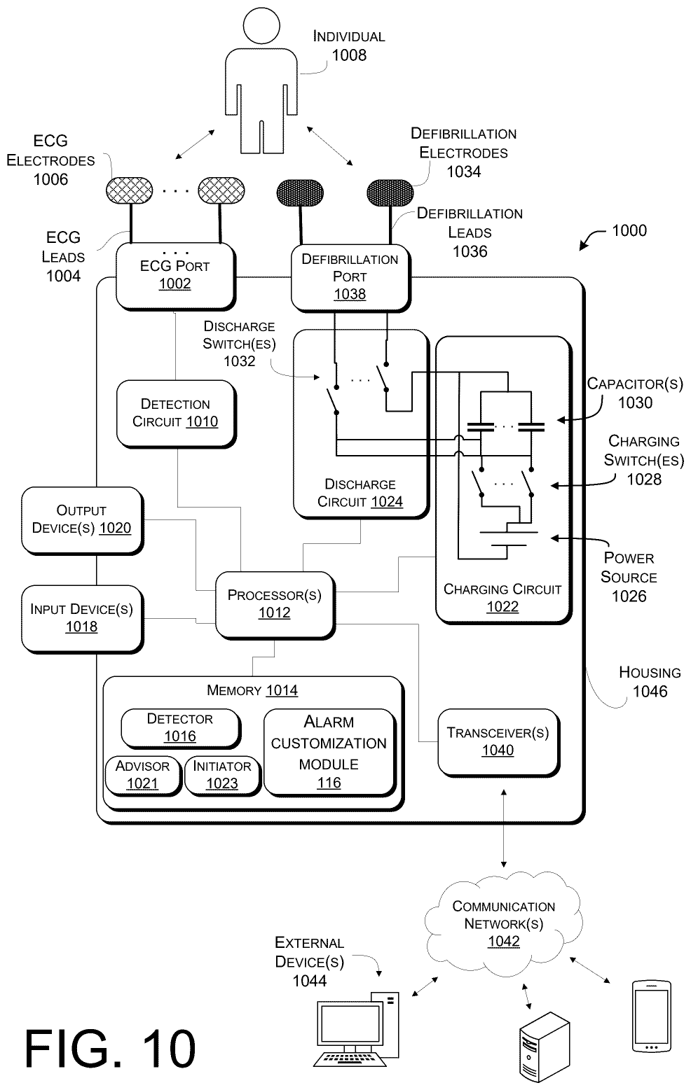

FIG. 10 illustrates an example of an external defibrillator 1000 configured to perform various functions described herein. For example, the external defibrillator 1000 is the medical device 106 described above and introduced in FIGS. 1A and 1B.

The external defibrillator 1000 includes an electrocardiogram (ECG) port 1002 connected to multiple ECG leads 1004. In some cases, the ECG leads 1004 are removeable from the ECG port 1002. For instance, the ECG leads 1004 are plugged into the ECG port 1002. The ECG leads 1004 are connected to ECG electrodes 1006, respectively. In various implementations, the ECG electrodes 1006 are disposed on different locations on an individual 1008. A detection circuit 1010 is configured to detect relative voltages between the ECG electrodes 1006. These voltages are indicative of the electrical activity of the heart of the individual 1008.

In various implementations, the ECG electrodes 1006 are in contact with the different locations on the skin of the individual 1008. In some examples, a first one of the ECG electrodes 1006 is placed on the skin between the heart and right arm of the individual 1008, a second one of the ECG electrodes 1006 is placed on the skin between the heart and left arm of the individual 1008, and a third one of the ECG electrodes 1006 is placed on the skin between the heart and a leg (either the left leg or the right leg) of the individual 1008. In these examples, the detection circuit 1010 is configured to measure the relative voltages between the first, second, and third ECG electrodes 1006. Respective pairings of the ECG electrodes 1006 are referred to as “leads,” and the voltages between the pairs of ECG electrodes 1006 are known as “lead voltages.” In some examples, more than three ECG electrodes 1006 are included, such that 5-lead or 12-lead ECG signals are detected by the detection circuit 1010.

The detection circuit 1010 includes at least one analog circuit, at least one digital circuit, or a combination thereof. The detection circuit 1010 receives the analog electrical signals from the ECG electrodes 1006, via the ECG port 1002 and the ECG leads 1004. In some cases, the detection circuit 1010 includes one or more analog filters configured to filter noise and/or artifact from the electrical signals. The detection circuit 1010 includes an analog-to-digital (ADC) in various examples. The detection circuit 1010 generates a digital signal indicative of the analog electrical signals from the ECG electrodes 1006. This digital signal can be referred to as an “ECG signal” or an “ECG.”

In some cases, the detection circuit 1010 further detects an electrical impedance between at least one pair of the ECG electrodes 1006. For example, the detection circuit 1010 includes, or otherwise controls, a power source that applies a known voltage (or current) across a pair of the ECG electrodes 1006 and detects a resultant current (or voltage) between the pair of the ECG electrodes 1006. The impedance is generated based on the applied signal (voltage or current) and the resultant signal (current or voltage). In various cases, the impedance corresponds to respiration of the individual 1008, chest compressions performed on the individual 1008, and other physiological states of the individual 1008. In various examples, the detection circuit 1010 includes one or more analog filters configured to filter noise and/or artifact from the resultant signal. The detection circuit 1010 generates a digital signal indicative of the impedance using an ADC. This digital signal can be referred to as an “impedance signal” or an “impedance.”

The detection circuit 1010 provides the ECG signal and/or the impedance signal one or more processors 1012 in the external defibrillator 1000. In some implementations, the processor(s) 1012 includes a CPU, a GPU, both CPU and GPU, or other processing unit or component known in the art. In some examples, the processor(s) 1012 is/are the same as or similar to the processor(s) 112 described above.

The processor(s) 1012 is operably connected to memory 1014. In some examples, the memory 1014 is the same as or similar to the memory 114 described above. In various implementations, the memory 1014 is volatile (such as random access memory (RAM)), non-volatile (such as read only memory (ROM), flash memory, etc.) or some combination of the two. The memory 1014 stores instructions that, when executed by the processor(s) 1012, causes the processor(s) 1012 to perform various operations. In various examples, the memory 1014 stores methods, threads, processes, applications, objects, modules, any other sort of executable instruction, or a combination thereof. In some cases, the memory 1014 stores files, databases, or a combination thereof. In some examples, the memory 1014 includes, but is not limited to, RAM, ROM, electrically erasable programmable read-only memory (EEPROM), flash memory, or any other memory technology. In some examples, the memory 1014 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1012 and/or the external defibrillator 1000. In some cases, the memory 1014 at least temporarily stores the ECG signal and/or the impedance signal.

In various examples, the memory 1014 includes a detector 1016, which causes the processor(s) 1012 to determine, based on the ECG signal and/or the impedance signal, whether the individual 1008 is exhibiting a particular heart rhythm. For instance, the processor(s) 1012 determines whether the individual 1008 is experiencing a shockable rhythm that is treatable by defibrillation. Examples of shockable rhythms include ventricular fibrillation (VF) and ventricular tachycardia (V-Tach). In some examples, the processor(s) 1012 determines whether any of a variety of different rhythms (e.g., asystole, sinus rhythm, atrial fibrillation (AF), etc.) are present in the ECG signal.

The processor(s) 1012 is operably connected to one or more input devices 1018 and one or more output devices 1020. In some examples, the input device(s) 1018 is/are the same as or similar to the input device(s) 120 described above. In some examples, the output device(s) 1020 is/are the same as or similar to the output device(s) 122 described above. Collectively, the input device(s) 1018 and the output device(s) 1020 function as an interface between a user and the defibrillator 1000. The input device(s) 1018 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 1020 includes at least one of a display, a speaker, a haptic output device, a printer, or any combination thereof. In various examples, the processor(s) 1012 causes a display among the input device(s) 1018 to visually output a waveform of the ECG signal and/or the impedance signal. In some implementations, the input device(s) 1018 includes one or more touch sensors, the output device(s) 1020 includes a display screen, and the touch sensor(s) are integrated with the display screen. Thus, in some cases, the external defibrillator 1000 includes a touchscreen configured to receive user input signal(s) and visually output physiological parameters, such as the ECG signal and/or the impedance signal.

In some examples, the memory 1014 includes an advisor 1021, which, when executed by the processor(s) 1012, causes the processor(s) 1012 to generate advice and/or control the output device(s) 1020 to output the advice to a user (e.g., a rescuer). In some examples, the processor(s) 1012 provides, or causes the output device(s) 1020 to provide, an instruction to perform CPR on the individual 1008. In some cases, the processor(s) 1012 evaluates, based on the ECG signal, the impedance signal, or other physiological parameters, CPR being performed on the individual 1008 and causes the output device(s) 1020 to provide feedback about the CPR in the instruction. According to some examples, the processor(s) 1012, upon identifying that a shockable rhythm is present in the ECG signal, causes the output device(s) 1020 to output an instruction and/or recommendation to administer a defibrillation shock to the individual 1008.

The memory 1014 also includes an initiator 1023 which, when executed by the processor(s) 1012, causes the processor(s) 1012 to control other elements of the external defibrillator 1000 in order to administer a defibrillation shock to the individual 1008. In some examples, the processor(s) 1012 executing the initiator 1023 selectively causes the administration of the defibrillation shock based on determining that the individual 1008 is exhibiting the shockable rhythm and/or based on an input from a user (received, e.g., by the input device(s) 1018. In some cases, the processor(s) 1012 causes the defibrillation shock to be output at a particular time, which is determined by the processor(s) 1012 based on the ECG signal and/or the impedance signal.

The processor(s) 1012 is operably connected to a charging circuit 1022 and a discharge circuit 1024. In various implementations, the charging circuit 1022 includes a power source 1026, one or more charging switches 1028, and one or more capacitors 1030. The power source 1026 includes, for instance, a battery. The processor(s) 1012 initiates a defibrillation shock by causing the power source 1026 to charge at least one capacitor among the capacitor(s) 1030. For example, the processor(s) 1012 activates at least one of the charging switch(es) 1028 in the charging circuit 1022 to complete a first circuit connecting the power source 1026 and the capacitor to be charged. Then, the processor(s) 1012 causes the discharge circuit 1024 to discharge energy stored in the charged capacitor across a pair of defibrillation electrodes 1034, which are in contact with the individual 1008. For example, the processor(s) 1012 deactivates the charging switch(es) 1028 completing the first circuit between the capacitor(s) 1030 and the power source 1026, and activates one or more discharge switches 1032 completing a second circuit connecting the charged capacitor 1030 and at least a portion of the individual 1008 disposed between defibrillation electrodes 1034.

The energy is discharged from the defibrillation electrodes 1034 in the form of a defibrillation shock. For example, the defibrillation electrodes 1034 are connected to the skin of the individual 1008 and located at positions on different sides of the heart of the individual 1008, such that the defibrillation shock is applied across the heart of the individual 1008. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 1032 are controlled by the processor(s) 1012, for example. In various implementations, the defibrillation electrodes 1034 are connected to defibrillation leads 1036. The defibrillation leads 1036 are connected to a defibrillation port 1038, in implementations. According to various examples, the defibrillation leads 1036 are removable from the defibrillation port 1038. For example, the defibrillation leads 1036 are plugged into the defibrillation port 1038.

In various implementations, the processor(s) 1012 is operably connected to one or more transceivers 1040 that transmit and/or receive data over one or more communication networks 1042. In some examples, the transceiver(s) 1040 is/are the same as or similar to the transceiver(s) 128 described above. In an example, the transceiver(s) 1040 includes a network interface card (NIC), a network adapter, a local area network (LAN) adapter, or a physical, virtual, or logical address to connect to the various external devices and/or systems. In various examples, the transceiver(s) 1040 includes any sort of wireless transceivers capable of engaging in wireless communication (e.g., radio frequency (RF) communication). For example, the communication network(s) 1042 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1040 includes other wireless modems, such as a modem for engaging in WI-FI®, WIGIG®, WIMAX®, BLUETOOTH®, NFC, radio frequency identification (RFID), or infrared communication over the communication network(s) 1042.

The defibrillator 1000 is configured to transmit and/or receive data (e.g., ECG data, impedance data, data indicative of one or more detected heart rhythms of the individual 1008, data indicative of one or more defibrillation shocks administered to the individual 1008, etc.) with one or more external devices 1044 via the communication network(s) 1042. The external devices 1044 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 1042. In some examples, the external device(s) 1044 is located remotely from the defibrillator 1000, such as at a remote clinical environment (e.g., a hospital). According to various implementations, the processor(s) 1012 causes the transceiver(s) 1040 to transmit data to the external device(s) 1044. In some cases, the transceiver(s) 1040 receives data from the external device(s) 1044 and the transceiver(s) 1040 provide the received data to the processor(s) 1012 for further analysis.

In some cases, the external device(s) 1044 include one or more medical devices. According to various implementations, the memory 1014 further includes the aforementioned alarm customization module 116 which, when executed by the processor(s) 1012, causes the processor(s) 1012 to perform any of the techniques and/or processes described herein.

In various implementations, the external defibrillator 1000 also includes a housing 1046 that at least partially encloses other elements of the external defibrillator 1000. For example, the housing 1046 encloses the detection circuit 1010, the processor(s) 1012, the memory 1014, the charging circuit 1022, the transceiver(s) 1040, or any combination thereof. In some cases, the input device(s) 1018 and output device(s) 1020 extend from an interior space at least partially surrounded by the housing 1046 through a wall of the housing 1046. In various examples, the housing 1046 acts as a barrier to moisture, electrical interference, and/or dust, thereby protecting various components in the external defibrillator 1000 from damage.

In some implementations, the external defibrillator 1000 is an automated external defibrillator (AED) operated by an untrained user (e.g., a bystander, layperson, etc.) and can be operated in an automatic mode. In automatic mode, the processor(s) 1012 automatically identifies a rhythm in the ECG signal, makes a decision whether to administer a defibrillation shock, charges the capacitor(s) 1030, discharges the capacitor(s) 1030, or any combination thereof. In some cases, the processor(s) 1012 controls the output device(s) 1020 to output (e.g., display) a simplified user interface to the untrained user. For example, the processor(s) 1012 refrains from causing the output device(s) 1020 to display a waveform of the ECG signal and/or the impedance signal to the untrained user, in order to simplify operation of the external defibrillator 1000.

In some examples, the external defibrillator 1000 is a monitor-defibrillator utilized by a trained user (e.g., a clinician, an emergency responder, etc.) and can be operated in a manual mode or the automatic mode. When the external defibrillator 1000 operates in manual mode, the processor(s) 1012 cause the output device(s) 1020 to display a variety of information that may be relevant to the trained user, such as waveforms indicating the ECG data and/or impedance data, notifications about detected heart rhythms, and the like.

### EXAMPLE CLAUSES

1. A medical device including: a sensor configured to detect a physiological parameter; an input device configured to receive contextual data indicative of a context of an emergency scene where a patient is located; an output device; and a processor configured to: infer a medical condition of the patient by analyzing the contextual data; customize a set of alarms to obtain a customized set of alarms tailored to the medical condition of the patient; receive, via the sensor, the physiological parameter of the patient; compare the physiological parameter of the patient to a threshold associated with the customized set of alarms; in response to comparing the physiological parameter to the threshold, generate an alarm of the customized set of alarms, wherein the alarm is associated with the medical condition; and cause the alarm to be output via the output device.
2. The medical device of clause 1, wherein: the contextual data includes location data representing a location of the emergency scene; and inferring the medical condition of the patient includes: accessing, from a datastore, an electronic medical record associated with a registered patient residing at the location; and determining, from information in the electronic medical record, that the registered patient has a history of the medical condition.
3. The medical device of clause 1 or 2, wherein: the contextual data includes audio data representing sound in an environment of the medical device while the medical device is located at the emergency scene; and inferring the medical condition of the patient includes: processing the audio data to determine a sound recognition result indicative of agonal breathing; and determining, from the sound recognition result, that the patient is likely experiencing a respiratory problem.
4. The medical device of any of clauses 1 to 3, wherein customizing the set of alarms includes changing the threshold used for generating the alarm.
5. The medical device of any of clauses 1 to 4, wherein customizing the set of alarms includes disabling or muting one or more additional alarms of the set of alarms that are (i) not associated with the medical condition, or (ii) associated with the medical condition, but not beneficial for continuing treatment.
6. A medical device including: an input device configured to receive contextual data associated with an emergency scene where a patient is located; an output device; and a processor configured to: determine a characteristic of the patient by analyzing the contextual data; customize a set of alarms to the characteristic of the patient to obtain a customized set of alarms; generate an alarm of the customized set of alarms by analyzing a physiological parameter of the patient; and cause the alarm to be output via the output device.
7. The medical device of clause 6, wherein: the contextual data includes location data representing a location of the emergency scene; and determining the characteristic of the patient includes: accessing, from a datastore, an electronic medical record associated with a registered patient residing at the location; and determining the characteristic of the patient by analyzing information in the electronic medical record.
8. The medical device of clause 6 or 7, wherein: the contextual data includes audio data representing sound in an environment of the medical device while the medical device is located at the emergency scene; and determining the characteristic of the patient includes: processing the audio data to determine a sound recognition result; and determining the characteristic of the patient by analyzing the sound recognition result.
9. The medical device of any of clauses 6 to 8, wherein: the contextual data includes image data representing the patient at the emergency scene; and determining the characteristic of the patient includes: processing the image data to determine an image recognition result; and determining the characteristic of the patient by analyzing the image recognition result.
10. The medical device of any of clauses 6 to 9, wherein: the contextual data is indicative of a size of the patient; and the characteristic of the patient is that the patient is obese.
11. The medical device of any of clauses 6 to 10, wherein: the contextual data includes environmental data representing a condition of an environment of the emergency scene; and the condition of the environment is associated with air temperature, air quality, or flooding in the environment.
12. The medical device of any of clauses 6 to 11, wherein customizing the set of alarms includes prioritizing output of the alarm over one or more additional alarms of the set of alarms.
13. The medical device of any of clauses 6 to 12, wherein: the output device includes a display; and customizing the set of alarms includes changing a color, a size, or a shape of a visual indicator that is to be presented via a user interface on the display.
14. A method including: receiving, by a medical device, contextual data associated with an emergency scene where a patient is located; determining, by the medical device analyzing the contextual data, a characteristic of the patient; customizing, by the medical device, a set of alarms to the characteristic of the patient to obtain a customized set of alarms; generating, by the medical device analyzing a physiological parameter of the patient, an alarm of the customized set of alarms; and causing, by the medical device, the alarm to be output.
15. The method of clause 14, wherein the customizing the set of alarms includes muting one or more additional alarms of the set of alarms.
16. The method of clause 14 or 15, wherein the analyzing the contextual data includes: providing the contextual data as input to a trained artificial intelligence model; and receiving the characteristic of the patient as output from the trained artificial intelligence model.
17. The method of any of clauses 14 to 16, further including: detecting, by the medical device, a presence of an additional medical device in a vicinity of the medical device; in response to the causing the alarm to be output, sending, via a transceiver of the medical device, control data to the additional medical device to cause the additional medical device to monitor or treat the patient; and in response to the sending the control data to the additional medical device, causing, by the medical device, the alarm to cease being output.
18. The method of any of clauses 14 to 17, further including: receiving, by the medical device, medication data representing a medication administered to the patient at the emergency scene; presenting, via a touch-sensitive display of the medical device, a potential alarm list that includes a subset of the customized set of alarms, wherein the subset is associated with the medication; receiving, via the touch-sensitive display, one or more selections of one or more alarms in the potential alarm list; and in response to the receiving of the one or more selections of the one or more alarms, disabling, by the medical device, the one or more alarms.
19 The method of any of clauses 14 to 18, further including, prior to arrival of the medical device at the emergency scene: causing an alarm configuration user interface to be presented via a display of a user device, wherein the alarm configuration user interface allows a user to preconfigure the set of alarms that the medical device is configured to output at emergency scenes; receiving one or more selections associated with the alarm configuration user interface; and causing the medical device to preconfigure the set of alarms based on the one or more selections.
20. The method of clause 19, wherein the one or more selections include one or more first selections, the method further including: receiving one or more second selections associated with the alarm configuration user interface; and causing, based on the one or more first selections, the user device to output the set of alarms in accordance with the one or more first selections as a simulation of the medical device outputting the set of alarms.
21. A medical device including: a sensor configured to detect a physiological parameter; a transceiver configured to receive, from a wearable device worn by a patient located at an emergency scene, a communication signal indicating a heart rate of the patient; an output device; and a processor configured to: customize a set of alarms to obtain a customized set of alarms tailored to the heart rate of the patient; receive, via the sensor, the physiological parameter of the patient; compare the physiological parameter of the patient to a threshold associated with the customized set of alarms; in response to comparing the physiological parameter to the threshold, generate an alarm of the customized set of alarms; and cause the alarm to be output via the output device.
22. The medical device of clause 21, wherein customizing the set of alarms includes prioritizing output of the alarm over one or more additional alarms of the set of alarms.
23 The medical device of clause 21 or 22, wherein customizing the set of alarms includes changing the threshold used for generating the alarm.
24. The medical device of any of clauses 21 to 23, wherein the transceiver is further configured to send, to the wearable device, authentication data for authenticating the medical device prior to receiving the communication signal.
25. The medical device of any of clauses 21 to 24, wherein: the communication signal includes encrypted data indicating the heart rate of the patient; and the processor is further configured to decrypt the encrypted data prior to customizing the set of alarms.
26. The medical device of any of clauses 21 to 25, wherein the wearable device is a smart watch.
27. A medical device including: a transceiver configured to receive, from a patient device of a patient located at an emergency scene, device data indicative of a first physiological parameter of the patient; an output device; and a processor configured to: customize a set of alarms to the first physiological parameter of the patient to obtain a customized set of alarms; generate an alarm of the customized set of alarms by analyzing a second physiological parameter of the patient; and cause the alarm to be output via the output device.
28 The medical device of clause 27, wherein customizing the set of alarms includes prioritizing output of the alarm over one or more additional alarms of the set of alarms.
29. The medical device of clause 27 or 28, wherein customizing the set of alarms includes changing a threshold used for generating the alarm.
30 The medical device of any of clauses 27 to 29, wherein the transceiver is further configured to send, to the patient device, authentication data for authenticating the medical device prior to receiving the device data.
31. The medical device of any of clauses 27 to 30, wherein: the device data is encrypted; and the processor is further configured to decrypt the device data prior to customizing the set of alarms.
32. The medical device of any of clauses 27 to 31, wherein the device data is received wirelessly using a wireless communication protocol.
33 The medical device of any of clauses 27 to 32, wherein the patient device is a wearable device, an implantable device, or a home health care device.
34. The medical device of clause 33, wherein the home health care device is an insulin pump.
35. A method including: receiving, by a medical device, from a patient device of a patient located at an emergency scene, device data indicative of a first physiological parameter of the patient; customizing, by the medical device, a set of alarms to the first physiological parameter of the patient to obtain a customized set of alarms; generating, by the medical device analyzing a second physiological parameter of the patient, an alarm of the customized set of alarms; and causing, by the medical device, the alarm to be output.
36. The method of clause 35, wherein the customizing the set of alarms includes prioritizing output of the alarm over one or more additional alarms of the set of alarms.
37. The method of clause 35 or 36, wherein the customizing the set of alarms includes changing a threshold used for the generating the alarm.
38. The method of any of clauses 35 to 37, further including sending, by the medical device, to the patient device, authentication data for authenticating the medical device prior to the receiving the device data.
39 The method of any of clauses 35 to 38, wherein: the device data is encrypted; and the method further includes decrypting the device data prior to the customizing the set of alarms.
40. The method of any of clauses 35 to 39, wherein the patient device is a wearable device, an implantable device, or a home health care device.

While the example clauses described above are described with respect to one particular implementation, it should be understood that, in the context of this document, the content of the example clauses can also be implemented via a method, device, system, computer-readable medium, and/or another implementation. Additionally, any one of example clauses 1-40 may be implemented alone or in combination with any other of the example clauses 1-40.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A medical device comprising:
a sensor configured to detect a physiological parameter;
an input device configured to receive contextual data indicative of a context of an emergency scene where a patient is located;
an output device; and
a processor configured to:
infer a medical condition of the patient by analyzing the contextual data;
customize a set of alarms to obtain a customized set of alarms tailored to the medical condition of the patient;
receive, via the sensor, the physiological parameter of the patient;
compare the physiological parameter of the patient to a threshold associated with the customized set of alarms;
in response to comparing the physiological parameter to the threshold, generate an alarm of the customized set of alarms, wherein the alarm is associated with the medical condition; and
cause the alarm to be output via the output device.

2. The medical device of claim 1, wherein:
the contextual data comprises location data representing a location of the emergency scene; and
inferring the medical condition of the patient comprises:
accessing, from a datastore, an electronic medical record associated with a registered patient residing at the location; and
determining, from information in the electronic medical record, that the registered patient has a history of the medical condition.

3. The medical device of claim 1, wherein:
the contextual data comprises audio data representing sound in an environment of the medical device while the medical device is located at the emergency scene; and
inferring the medical condition of the patient comprises:
processing the audio data to determine a sound recognition result indicative of agonal breathing; and
determining, from the sound recognition result, that the patient is likely experiencing a respiratory problem.

4. The medical device of claim 1, wherein customizing the set of alarms comprises changing the threshold used for generating the alarm.

5. The medical device of claim 1, wherein customizing the set of alarms comprises disabling or muting one or more additional alarms of the set of alarms that are (i) not associated with the medical condition, or (ii) associated with the medical condition, but not beneficial for continuing treatment.

6. A medical device comprising:
an input device configured to receive contextual data associated with an emergency scene where a patient is located;
an output device; and
a processor configured to:
determine a characteristic of the patient by analyzing the contextual data;
customize a set of alarms to the characteristic of the patient to obtain a customized set of alarms;
generate an alarm of the customized set of alarms by analyzing a physiological parameter of the patient; and
cause the alarm to be output via the output device.

7. The medical device of claim 6, wherein:
the contextual data comprises location data representing a location of the emergency scene; and
determining the characteristic of the patient comprises:
accessing, from a datastore, an electronic medical record associated with a registered patient residing at the location; and
determining the characteristic of the patient by analyzing information in the electronic medical record.

8. The medical device of claim 6, wherein:
the contextual data comprises audio data representing sound in an environment of the medical device while the medical device is located at the emergency scene; and
determining the characteristic of the patient comprises:
processing the audio data to determine a sound recognition result; and
determining the characteristic of the patient by analyzing the sound recognition result.

9. The medical device of claim 6, wherein:
the contextual data comprises image data representing the patient at the emergency scene; and
determining the characteristic of the patient comprises:
processing the image data to determine an image recognition result; and
determining the characteristic of the patient by analyzing the image recognition result.

10. The medical device of claim 6, wherein:
the contextual data is indicative of a size of the patient; and
the characteristic of the patient is that the patient is obese.

11. The medical device of claim 6, wherein:
the contextual data comprises environmental data representing a condition of an environment of the emergency scene; and
the condition of the environment is associated with air temperature, air quality, or flooding in the environment.

12. The medical device of claim 6, wherein customizing the set of alarms comprises prioritizing output of the alarm over one or more additional alarms of the set of alarms.

13. The medical device of claim 6, wherein:
the output device comprises a display; and
customizing the set of alarms comprises changing a color, a size, or a shape of a visual indicator that is to be presented via a user interface on the display.

14. A method comprising:
receiving, by a medical device, contextual data associated with an emergency scene where a patient is located;
determining, by the medical device analyzing the contextual data, a characteristic of the patient;
customizing, by the medical device, a set of alarms to the characteristic of the patient to obtain a customized set of alarms;
generating, by the medical device analyzing a physiological parameter of the patient, an alarm of the customized set of alarms; and
causing, by the medical device, the alarm to be output.

15. The method of claim 14, wherein the customizing the set of alarms comprises muting one or more additional alarms of the set of alarms.

16. The method of claim 14, wherein the analyzing the contextual data comprises:
providing the contextual data as input to a trained artificial intelligence model; and
receiving the characteristic of the patient as output from the trained artificial intelligence model.

17. The method of claim 14, further comprising:
detecting, by the medical device, a presence of an additional medical device in a vicinity of the medical device;
in response to the causing the alarm to be output, sending, via a transceiver of the medical device, control data to the additional medical device to cause the additional medical device to monitor or treat the patient; and
in response to the sending the control data to the additional medical device, causing, by the medical device, the alarm to cease being output.

18. The method of claim 14, further comprising:
receiving, by the medical device, medication data representing a medication administered to the patient at the emergency scene;
presenting, via a touch-sensitive display of the medical device, a potential alarm list that includes a subset of the customized set of alarms, wherein the subset is associated with the medication;
receiving, via the touch-sensitive display, one or more selections of one or more alarms in the potential alarm list; and
in response to the receiving of the one or more selections of the one or more alarms, disabling, by the medical device, the one or more alarms.

19. The method of claim 14, further comprising, prior to arrival of the medical device at the emergency scene:
causing an alarm configuration user interface to be presented via a display of a user device, wherein the alarm configuration user interface allows a user to preconfigure the set of alarms that the medical device is configured to output at emergency scenes;
receiving one or more selections associated with the alarm configuration user interface; and
causing the medical device to preconfigure the set of alarms based on the one or more selections.

20. The method of claim 19, wherein the one or more selections comprise one or more first selections, the method further comprising:
receiving one or more second selections associated with the alarm configuration user interface; and
causing, based on the one or more first selections, the user device to output the set of alarms in accordance with the one or more first selections as a simulation of the medical device outputting the set of alarms.
